(19) United States
(12) Patent Application Publication
(10) Pub. No.: US 2019/0125349 A1
(43) Pub. Date: May 2, 2019

(54) CONNECTOR DEVICE

(71) Applicant: MEDICAL CONNECTION TECHNOLOGY - MEDICONNTECH - M.C.T. LTD., Beit-Zera (IL)

(72) Inventors: Shay KAHANA, Beit-Zera (IL); Zvi NITZAN, Moshav Zofit (IL)

(21) Appl. No.: 16/085,579

(22) PCT Filed: Mar. 14, 2017
(86) PCT No.: PCT/IL2017/050323

§ 371 Date: Sep. 16, 2018

Related U.S. Application Data
(60) Provisional application No. 62/309,475, filed on Mar. 17, 2016.
(60) Provisional application No. 62/310,748, filed on Mar. 20, 2016.
(60) Provisional application No. 62/358,072, filed on Jul. 4, 2016.
(60) Provisional application No. 62/453,015, filed on Feb. 1, 2017.

(51) Int. Cl. A61B 17/11

(52) U.S. Cl. CPC A61B 17/11; A61B 2017/1132; A61B 2017/00867; A61B 2017/1139; A61B 2017/1135

(57) ABSTRACT

The present invention provides a device for connection of a biological structure with at least one other structure, the device including a first tubular member and a second tubular member and at least one arm for connecting the first tubular member to the second tubular member. The first tubular member includes a first opening at a proximal extremity of the tubular member, a second opening at a distal extremity of the tubular member and a central cavity within the body of the tubular member for accommodating the biological structure. The second tubular member includes a first opening at a proximal extremity of the tubular member, a second opening at a distal extremity of the tubular member and a central cavity within the body of the tubular member. The present invention further provides methods of connecting one or more structures, such as biological structures and grafts.

This application claims priority to U.S. Provisional Patent Application No. 62/309,475 filed on Mar. 17, 2016 and U.S. Provisional Patent Application No. 62/310,748 filed on Mar. 20, 2016 and U.S. Provisional Patent Application No. 62/358,072 filed on Jul. 4, 2016 and U.S. Provisional Patent Application No. 62/453,015 filed on Feb. 1, 2017 and incorporated herein by reference in their entirety.

### FIELD OF THE INVENTION

The present invention relates to a connector, a connector attachment means and a method of use thereof. Moreover, the present invention is of a connector for joining body tissues.

### BACKGROUND OF THE INVENTION

Vascular anastomosis is the procedure of connecting together blood conduits, such as vessels, prostheses or grafts. The main aim of performing a vascular anastomosis is to achieve maximal patency rates with minimal damage to the vessel walls. Devices, methods and adjuvants used to hold body tissues together include sutures, glue, staples, clips, clamps, rings, stents, suturing, gluing, stapling, clipping, laser welding and combinations thereof.

The most common device for holding body tissues together after injury or during surgery is surgical suture. Suturing is a cheap technique and is a method of choice for joining body tissue, such as in vascular anastomosis, but the technique suffers from disadvantages. Suturing is slow, prolonging surgery, which results in more time the patient must be anaesthetized and less available time for other patients in the operating theatre. Suturing is also not ideal in small blood vessels. Additional problems include leaking from the gaps between each stitch and damage to the vascular wall.

Other methods may be quicker than suturing, but are not ideal. Adhesives are difficult to use in anastomosis of small blood vessels, may take time to work, may not be strong enough to hold together certain biological structures for the needed time period and may cause toxicity, leakage and aneurysm formation. Staples suffer from the disadvantage that they need to be removed and may cause more stenosis than sutures. Stents may result in early occlusion. Laser welding is costly, necessitating specialized surgical skills and may have reduced strength in larger-sized vessels. Clips are problematic in long term joining of vessels of larger diameter where the pressure is too high. Problems associated with available rings include resulting rigidity and a non-compliant anastomosis.

It would therefore be desirable to have a device and method for joining body tissues, which would be quick to use, provide uniform connection and patency without the problem of narrowing the lumen and rigidity. It would also be advantageous if the method was of low cost and did not require unusual experience or skill. The present invention provides such a device and method.

### SUMMARY

The invention may have several aspects. One aspect is a device of the present invention. The device may be for connection of a first structure with at least one other structure. The device may include a first tubular member, a second tubular member corresponding to the first tubular member and at least one arm for connecting the first tubular member to the second tubular member. The first tubular member may include a proximal opening at a proximal extremity of the tubular member, a distal opening at a distal extremity of the tubular member and a central cavity within the body of the tubular member for accommodating the first structure. The second tubular member may include a proximal opening at a proximal extremity of the tubular member, a distal opening at a distal extremity of the tubular member and a central cavity within the body of the tubular member. At least one of the first structure and the at least one other structure is a biological structure.

In various embodiments of the device, the proximal opening of the first tubular member and the proximal opening of the second tubular member may be a uniform opening positioned ninety degrees to the longitudinal body of the tubular member. The proximal opening may be parallel to the distal opening. The proximal opening and the distal opening may be similarly sized and shaped. The central cavity of the second tubular member may be for accommodating a length of the at least one other structure to be connected.

In various embodiments of the device, the proximal opening of the first tubular member and the proximal opening of the second tubular member may be a non-uniform opening, wherein at least part of the opening is not positioned, ninety degrees to the longitudinal body of the tubular member and wherein at least part of the opening is carved out of the body of the tubular member. The proximal opening of the first tubular member may correspond to the proximal opening of the second tubular member. The proximal opening of the first tubular member may not be parallel to the distal opening of the first tubular member.

In various embodiments of the device, the first tubular member and the second tubular member feature a plurality of spaced apart protrusions on the body of the tubular member for holding an end of a structure everted over the proximal extremity of the tubular member. The plurality of spaced apart protrusions may be adjacent to the proximal extremity of the tubular member and may protrude outwards away from an exterior surface of the tubular member and in a direction towards the distal extremity of the tubular member. The plurality of spaced apart protrusions may include spikes.

In various embodiments of the device the at least one arm may be a plurality of arms. The at least one arm may include an arcuate arm. The at least one arm may feature an extension, the extension including a first end and a second end. The at least one arm may be freely attached to one of the first tubular member and the second tubular member by attachment of the first end of the at least one arm to an attachment point adjacent to the distal extremity of the one of the first tubular member and the second tubular member, to facilitate a tubular member with at least one arm, such that the extension and the second end of the extension of the at least one arm is restrictably moveable about the attached first end at the attachment point. The first end of the at least one arm may be fixedly attached to the one of the first tubular member and the second tubular member. The first end of the at least one arm may be detachably attached to the one of the first tubular member and the second tubular member. A plurality of the at least one arms may be attached in spaced apart relation about the circumference of the distal extremity of the one of the first tubular member and the second tubular member. The second end of the at least one arm may include at least one attachment member adapted to attach the first tubular member to the second tubular member.

In various embodiments of the device, the at least one arm may include an arch. The diameter of the arch may be equal to at least the combined length of the first tubular member and the second tubular member. The flexibility of the arch may facilitate a diameter equal to at least the combined length of the first tubular member and the length of the second tubular member and the length of everted structures protruding over the proximal extremity of the first tubular member and the second tubular member. The arm may be adapted to attach to the corresponding tubular member by the tension of the arch. The attachment member may include a hook positioned at the free end of the at least one arm. The hook may include at least one prong positioned at an angle to the arch to facilitate hooking the distal end of the corresponding other tubular member of the device. The at least one prong may include at least one protrusion. The at least one prong may feature a length of prong, a first end of the prong fixed to the free end of the at, least one arm and a free end of the prong. The at least one protrusion may feature a first at least one protrusion, positioned at the free end of the at least one prong, the first at least one protrusion, protruding at an angle to the length of prong, angled towards the first attached end of the arm and configured to hook the distal end of the corresponding other tubular member of the device and to attach into the cavity of the corresponding other tubular member to detachably fix the moveable arm to the corresponding other tubular member of the device. The at least one protrusion may include a second at least one protrusion. The second at least one protrusion may protrude at an angle to the length of the prong, angled towards the first attached end of the arm. The second at least one protrusion may protrude from a position along the length of the prong spaced apart from the first at least one protrusion. The second at least one protrusion may be longer than the first at least one protrusion and may be configured to allow the first at least one protrusion to attach into the cavity of the corresponding tubular member of the device and to prevent displacement of an additional part of the arm from entering the cavity of the corresponding other tubular member. The corresponding other tubular member may include a corresponding at least one attachment means for attaching the at least one arm attachment members of the one of the first tubular member and the second tubular member with the fixed arm attachment member. The corresponding at least one attachment means may feature at least one of a portion of the cavity of the other tubular member, the distal edge of the other tubular member and a slot/s. The at least one arm may include at least one downward protruding member adapted to prevent non-unitary displacement of at least one of the first tubular member and the second tubular member in a unitary device, the unitary device including a connected first tubular member and second tubular member.

In various embodiments of the device the at least one arm may feature a plurality of arms and an arm of the plurality of arms may include a linear extension, the at least one attachment member featuring a ratchet means disposed along the length of the arm extension. The ratchet means may include a plurality of spaced apart unidirectional teeth. The plurality of arms may be fixed to the one of the first tubular member and the second tubular member to facilitate the plurality of arms inclined at an angle away from the body of the one of the first tubular member and the second tubular member in a direction towards the proximal end of the one of the first tubular member and the second tubular member, the plurality of arms overhanging, the proximal end of the one of the first tubular member and the second tubular member. The other of the first tubular member and second tubular member to which the plurality of arms is not fixed may include a plurality of corresponding means to the at least one attachment member. The plurality of corresponding means may feature a plurality of raised unidirectional slits. The plurality of unidirectional slits may be fixed in spaced relation to the body of the other of the first tubular member and the second tubular member to which the plurality of arms is not fixed, to correspond to a respective arm of the plurality of arms. The plurality of unidirectional slits may be elevated to a height below the height of the respective arm. Each of the plurality of elevated slits may be configured for receiving one of the plurality of arms through the slit and for preventing the arm from reverse displacement out of the slit and for promoting the arm to proceed through the slit until the proximal opening of the first tubular member and the proximal opening of the second tubular member or structures everted thereon are in contact.

In various embodiments of the device the at least one arm may feature a plurality of arms. An arm of the plurality of arms may include a linear extension. The at least one attachment member may include a plurality of openings disposed spaced apart along the length of the linear extension. The plurality of arms may be fixed to the one of the first tubular member and the second tubular member to facilitate the plurality of arms inclined at an angle away from the body of the tubular member with the at least one arm in a direction towards the proximal end of the tubular member with the at least one arm and overhanging the proximal end of the tubular member with the at least one arm. The other of the first tubular member and second tubular member to which the plurality of arms is not fixed may include a plurality of corresponding means to the at least one attachment member. The plurality of corresponding means may feature a plurality of protrusions spaced apart and fixed on the body of the other tubular member. The plurality of protrusions may be configured to fit into an opening of the plurality of openings on the linear extension of the arm and may be angled to prevent reverse displacement out of the opening. The plurality of protrusions may be fixed such that the free ends of the plurality of protrusions are positioned at an elevated height with respect to the corresponding arm of the plurality of arms.

In various embodiments of the device the device may be for end to end connection. The device may be for side to end connection. The first tubular member and the second tubular member may be made from at least one material selected from the group consisting of metal, plastic, nitinol, alloys of titanium and nickel, stainless steel, platinum, gold, silver, copper, zinc, silicone, ceramic, polytetrafluoroethylene (PTFE), polyethylene, urethane, nylon, polyester, polypropylene, fabric, gut and tissue graft and combinations thereof. An internal surface of the tubular member may be coated with at feast one layer of coating selected from the group consisting of an adhesive, a glue, a surgical glue, a fibrin sealant, a collagen based compound, a glutaraldehyde glue, a hydrogel, a dye, an antibiotic, an antibacterial, an anti-clotting, substance, a vitamin, a promoter of healing, a therapeutic agent, an anticlotting substance, a clotting substance, an antioxidant, an anti-inflammatory agent, an anesthetic agent, an anti-coagulant, an anti-restenosis agent, a thrombosis agent, an immunosuppressant agent and a movement retardation composition.

In various embodiments of the device, the first tubular member and the second tubular member may be split rings, which may feature a length of tubular member configured in a tubular conformation with a longitudinal split. The length of the tubular member may be furled to facilitate a reversibly expandable central cavity of the tubular member, the central cavity configured without a gap. The furl may be configured to expand according to the dimensions of an inserted structure. In various embodiments, the first tubular member and the second tubular member may be configured as complete rings. In various embodiments, the first tubular member and the second tubular member may include a plurality of interwoven lengths configured for reversible radial enlargement of the tubular member when a pushing force is applied to at least one end of the tubular member and reversible radial contraction when a pulling force is applied to at least one end of the tubular member.

An additional aspect is a device of the present invention for connection of a first structure with at least one other structure. The device may include a first tubular member. The first tubular member may include a first opening at a proximal extremity of the tubular member, a second opening at a distal extremity of the tubular member, a central cavity within the tubular member for accommodating a first structure, a plurality of spaced apart protrusions for holding an end of the first structure everted over the proximal extremity of the tubular member and at least one arm fixedly attached to the first tubular member. The device may include a second tubular member. The second tubular member may include a first opening at a proximal extremity of the tubular member, a second opening at a distal extremity of the tubular member, a central cavity within the tubular member for accommodating the at least one other structure sand a plurality of spaced apart protrusions for holding an end of the at least one other structure everted, over the proximal extremity of the tubular member. The at least one arm may include a first end and a second end. The at least one arm may be freely attached to the first tubular member by attachment of the first end of the at least one arm to the first tubular member adjacent to the distal extremity. The second end of the at least one arm may include at least one attachment member detachably attached to the distal extremity of the second tubular member facilitating connection of the first tubular member to the second tubular member.

The at least one arm may feature at least one of an arcuate arm and an inclined linear arm. The at least one attachment member may include at least one of at least one hook attached to the cavity of the second tubular member, a ratchet means inserted through an elevated slit of the second tubular member and a plurality of spaced apart openings through which a protrusion fixed on the second tubular member may be inserted. The first structure may be a biological structure. The first tubular member May accommodate the biological structure inserted in the cavity of the first tubular member and the end to be connected of the biological structure may be everted over the proximal extremity of the first tubular member. The second tubular member may accommodate the at least one other structure inserted in the cavity of the second tubular member and the end to be connected of the at least one other structure may be everted over the proximal extremity of the second tubular member, such that the everted end of the biological structure, is in contact with the everted end of the at least one other structure.

A further aspect is a connecting means of the present invention. The connecting means may be for connecting a first part and a second part of a device for end to end anastomosis. The device may be for external application to at least one biological structure. The first part of the device may include a first opening at a proximal end of-the device, the first opening proximal to the anastomosis connection and a second opening at a distal end of the device, the second opening distal to the anastomosis connection. The second part of the device may include a first opening at a proximal end of the device, the first opening proximal to the anastomosis connection and a second opening at a distal, end of the device, the second, opening distal to the anastomosis connection. The connecting means may include at least one flexible arched arm. The at least one flexible arched arm may include a first end and a second end. The first end may include attachment means to attach to the distal end of the fist part of the device and the second end may include attachment means to attach to the distal end of the second part of the device. The first end attachment means may include a clamp for damping to the distal end of the first part of the device, facilitating a clamped arm moveable about the clamped attachment. The second end attachment means may feature at least one prong positioned at an angle to the arch to facilitate hooking the distal end of the second part of the device. The at least one prong may include a length of prong, a free end and at least one protrusion. The at least one protrusion may feature a first at least one protrusion positioned at the free end of the at least one prong, protruding at an angle towards the first end of the arm and configured to hook the distal end of the second part of the device and to attach into a cavity of the second part of the device to detachably fix the moveable at least one arm to the second part of the device. The at least one protrusion may include a second at least one protrusion, fixed in a position along the length of the prong and protruding at art angle towards the first end of the arm. The second at least one protrusion may be spaced apart from the first at least one protrusion. The second at least one protrusion may be longer than the first at least one protrusion. The second at least one protrusion may be configured to prevent displacement of an additional part of the at least one arm from entering the cavity of the second part of the device.

Another aspect is a system of the present invention. The system may be a system for connecting a first end of a graft and a second end of a graft to a first end of a cut blood vessel and a second end of a cut blood vessel. The system may include a first device of the present invention wherein the first tubular member of the first device may be for accommodating the first end of the cut blood vessel and the second tubular member of the first device may be for accommodating the first end of the graft. The system may include a second device of the present invention, wherein the first tubular member of the second device may be for accommodating the second end of the graft and the second tubular member of the second device may be for accommodating the second end of the cut blood vessel.

A still further aspect is a device of the present invention for connection of a biological structure with at least one other structure. The device may include a first tubular member. The first tubular member may include a proximal opening at a proximal extremity of the tubular member, a distal opening at a distal extremity of the tubular member, a central cavity within the tubular member for accommodating the at least one other structure, a plurality of spaced apart protrusions for holding an end of the at least on other structure everted over the proximal extremity of the tubular member and at least one arm fixed about the distal extremity for connecting the first tubular member to a second tubular member. The device may include a second tubular member. The second tubular member may include a proximal opening at a proximal extremity of the tubular member, a distal opening at a distal extremity of the tubular member, the distal opening configured for placing about an opening of the biological structure, a central cavity within the tubular member and a plurality of spaced apart protrusions for holding a biological structure everted over the proximal extremity of the tubular member The proximal opening of the first tubular member may correspond to the proximal opening of the second tubular member and the proximal opening of the first tubular member and the proximal opening of the second tubular member may be shaped to provide an end to side connection. The shape of the proximal opening may facilitate the shape and angle of at least one of the end of the structure and the opening of the structure everted thereon.

An aspect is a device of the present invention. The device may be a device for connection of a biological structure with at least one other structure. The device may include a first tubular member. The first tubular member may include a proximal opening at a proximal extremity of the tubular member, a distal opening at a distal extremity of the tubular member and a central cavity within the body of the tubular member for accommodating the at least one other structure. The device may include a second tubular member, corresponding to the first tubular member. The second tubular member may include a proximal opening at a proximal extremity of the tubular member, a distal opening at a distal extremity of the tubular member, a central cavity within the body of the tubular member. The device may include at least one arm for connecting the first tubular member to the second tubular member. The proximal opening of the first tubular member may be configured for eversion of the at least one other structure about the proximal opening and the proximal opening of the second tubular member may be configured for eversion of the biological structure about the proximal opening. The first tubular member and the second tubular member may be configured for reversible radial expansion. The first tubular member and the second tubular member configured for reversible radial expansion may be at least one of split rings, complete rings, a plurality of interwoven lengths and furled rings. The proximal opening configured for eversion may include an even edge. The device may include a plurality of protrusions about the body of the first tubular member and the second tubular member for faxing an everted structure to the first tubular member and the second tubular member.

Another aspect is a system of the present invention for performing a bypass procedure to connect a graft to a first opening in an artery before a blockage, and to a second opening in an artery after a blockage. The system may include a first device for joining the first opening in the artery to a first end of the graft. The first deice may include a first tubular member. The first tubular member may include a proximal opening, at a proximal extremity of the tubular member, a distal opening at a distal extremity of the tubular member, a central cavity within the tubular member for accommodating the graft and a plurality of spaced apart protrusions for holding the first end of the graft over the proximal extremity of the tubular member. The first device may include a second tubular member corresponding to the first tubular member. The second tubular member may include a proximal opening at a proximal extremity of the tubular member, a distal opening at a distal extremity of the tubular member, a central cavity within the tubular member and a plurality of spaced apart protrusions for holding an edge of the first opening in the artery everted over the proximal extremity of the tubular member. The first device may include at least one arm for connecting the first tubular member to the second tubular member. The proximal opening of the first tubular member corresponds to the proximal opening of the second tubular member and wherein the proximal opening of the first tubular member and the proximal opening of the second tubular member are configured for an end to side connection. The system may include a second deuce for joining the second end of the graft to the second opening in the artery. The second device may include a first tubular member. The first tubular member may include a proximal opening at a proximal extremity of the tubular member, a distal opening at a distal extremity of the tubular member, a central cavity within the tubular member for accommodating the graft, and a plurality of spaced apart protrusions for holding the second end of the graft everted over the proximal extremity of the tubular member. The second device may include a second tubular member. The second tubular member may correspond to the first tubular member. The second tubular member may include a proximal opening at a proximal extremity of the tubular member, a distal opening at a distal extremity of the tubular member, a central cavity within the tubular member and a plurality of spaced apart protrusions for holding an edge of the second opening in the artery everted over the proximal extremity of the tubular member. The second device may include at least one arm for connecting the first tubular member to the second tubular member. The proximal opening of the first tubular member corresponds to the proximal opening of the second tubular member and wherein the proximal opening of the first tubular member and the proximal opening of the second tubular member are configured for an end to side connection. The at least one arm may be fixedly attached to part of the tubular member configured for accommodating the graft of the first device and the second device.

An additional aspect is a method of use of the device of the present invention. The method may be a method of connecting a first biological structure and a second at least one other structure. The first biological structure may be one part of a cut blood vessel and the second at least one other structure may be a second part of the cut blood vessel. The method of connecting a first biological structure and at least one other structure may include providing a first device. The first device may include a first tubular member and a second tubular member. The first tubular member may include a proximal opening at a proximal extremity of the tubular member, a distal opening at a distal extremity of the tubular member, a central cavity within the tubular member for accommodating the first biological structure, a plurality of spaced apart protrusions for holding an end of the first biological structure everted over the proximal extremity of the tubular member and at least one arm fixedly attached to the first tubular member. The second tubular member may include a proximal opening at a proximal extremity of the tubular member, a distal opening at a distal extremity of the tubular member, a central cavity within the tubular member for accommodating the second other structure, a plurality of spaced apart protrusions for holding an end of the second other structure evened over the proximal extremity of the tubular member. The method may include inserting into the cavity of the first tubular member, through the distal opening, the first biological structure or a part thereof The method may include everting the end of the first biological structure or part thereof over the opening at the proximal extremity of the first tubular member. The method may include fixing the everted end of the first biological structure or part thereof with the plurality of spaced apart protrusions on the first tubular member. The method may include inserting into the cavity, through the distal opening of the second tubular member, the second other structure to be joined to the first biological structure. The method may include everting the end of the second other structure over the opening at the proximal extremity of the second tubular member. The method may include fixing the everted end of the second other structure with the plurality of spaced apart protrusions on the second tubular member. The method may include contacting end to end the everted end of the first biological structure with the everted end of the second other structure. The method may include manipulating the at least one arm to attach to a corresponding means on the distal extremity of the second tubular member to connect the first tubular member to the second tubular member and to connect the end of the first biological structure with the end of the second other structure. The fixing may include spearing the everted end on the plurality of spaced apart protrusions. The manipulating may include at least one of pushing and pulling the at least one arm. The manipulating may include inserting a hook of the at least one arm through the distal opening of the second tubular member and into the cavity of the second tubular member. Manipulating may include inserting a toothed extension of the at least one arm through the corresponding means featuring at least one unidirectional slit fixed on the second tubular member. Manipulating may include pushing the at least one arm downwards to insert the toothed extension of the at least one arm through the at least one unidirectional slit, which is at a lower elevation than the toothed extension of the at least one arm. The corresponding means may include at least one spike and manipulating may include inserting the at least one spike fixed on the second tubular member into an opening of a plurality of spaced apart openings disposed along the length of the at least one arm.

In an embodiment the method may be a method for connecting a first end and a second end of a graft to a first end and a second end of a cut blood vessel. The method may include repeating the method of connecting a first biological structure and a second at least one other structure with a second device. In the first device the first biological structure may be the first end of the blood vessel and the second other structure may be the first end of the graft, and in the second device the first biological structure may be the second end of the blood vessel and the second other structure may be the second end of the graft.

An aspect is a method of using the device of the present invention for performing a bypass procedure. The method may include providing a system of the present invention for performing a bypass procedure to connect a graft to a first opening in an artery before a blockage and to a second opening in an artery after a blockage. The method may include inserting into the cavity of the first tubular member of the first device, through the distal opening, the first end of a graft blood vessel. The method may include everting the first end of the graft blood vessel over the proximal opening at the proximal extremity of the first tubular member of the first device. The method may include fixing the everted first end of the graft blood vessel with the plurality of spaced apart protrusions. The method may include placing the second tubular member of the first device on the artery and about the first opening in the artery. The method may include everting the edge of the first opening of the artery over the proximal opening at the proximal extremity of the second tubular member of the first device. The method may include fixing the everted edge of the first opening of the artery with the plurality of spaced apart protrusions. The method may include contacting end to side the everted end of the first end of the graft blood vessel with the everted edge of the first opening of the artery. The method may include manipulating the at least one arm of the first tubular member to attach to the distal extremity of the corresponding second tubular member of the first device to connect the first tubular member of the first device to the second tubular member of the first device and to connect the first end of the graft blood vessel with the first opening in the artery. The method may include inserting into the cavity of the first tubular member of the second device, through the distal opening, the second end of the graft. The method may include everting the second end of the graft over the proximal opening at the proximal extremity of the first tubular member of the second device. The method may include fixing the everted second end of the graft with the plurality of spaced apart protrusions. The method may include placing the second tubular member of the second device on the artery about the second opening in the artery. The method may include everting the edge, of the second opening in the artery over the proximal opening at the proximal extremity of the second tubular member of the second device. The method may include fixing the everted edge of the second opening in the artery with the plurality of spaced apart protrusions. The method may include contacting end to side the everted end of the second end of the graft with the everted edge of the second opening in the artery. The method may include manipulating the at least one arm of the fist tubular member to attach to the distal extremity of the corresponding second tubular member of the second device to connect the first tubular member of the second device to the second tubular member of the second device and to connect the second end of the graft with the second opening of the artery.

A further aspect is a method of production of a device of the present invention for connecting a biological structure with another structure. The method may include forming a first hollow tubular member with a first opening at a proximal extremity and a second opening at a distal extremity. The method may include forming a second be tubular member with a first opening at a proximal extremity and a second opening at a distal extremity. The method may include punching a plurality of spikes adjacent the proximal extremity of the first hollow tubular member and the second hollow tubular member. The method may include forming a plurality of arms. The method may include attaching a plurality of arms in spaced apart relation about the second distal extremity of the first hollow tubular member facilitating a plurality of arms extending from the distal extremity to beyond the proximal extremity of the first hollow tubular member. Attaching a plurality of arms may include welding the plurality of spaced apart arms to the first hollow tubular member. Forming may include forming a plurality of anus which may include forming a plurality of arched arms, wherein each of the plurality of arched arms includes a hook at the free end of the arm. Forming a plurality of arms may include forming a plurality of arched arms, which are adaptable to a diameter equal to at least the combined length of the first tubular member and the length of the second tubular member and the length of everted structures protruding over the proximal extremity of the first tubular member and the second tubular member. Forming a plurality of arms may include forming a plurality of linear arm extensions. Forming a plurality of arms may include forming a ratchet means along the linear arm extension and the method may further include fixing a plurality of slits in an elevated position about the distal extremity of the second hollow tubular member. Forming a plurality of arms may include forming a plurality of openings spaced apart along the length of the plurality of linear arm extensions.

### BRIEF DESCRIPTION OF THE DRAWINGS

The various features of the invention will best be appreciated by simultaneous reference to the description which tallows and the accompanying drawings and in which:

FIG. 1 shows a schematic view of an exemplary connector device according to air aspect of the present invention;

FIG. 2a shows a schematic view of an exemplary tubular connector device according to an aspect of the present invention;

FIG. 2b shows a schematic view of an exemplars tubular connector device with a split according to an aspect of the present invention;

FIG. 2c shows a schematic view of an exemplary furled tubular connector device according to an aspect of the present invention;

FIG. 2d shows a schematic view of an exemplary braided tubular connector device according to an aspect of the present invention;

FIG. 3 shows a schematic view of an exemplary tubular member with exemplary protrusions according to an aspect of the present invention;

FIG. 4a shows a schematic view of an exemplary connector arm according to an aspect of the present invention;

FIG. 4b shows a schematic view of an exemplary plurality of connector arms according to a aspect of the present invention;

FIG. 4c shows a schematic view of exemplary two parts of a connector device according to an aspect of the present invention;

FIG. 4d shows a schematic view of exemplary two parts of a connector device, joined according to an aspect of the present invention;

FIG. 5 shows a schematic view of a pair of tubular members with connector arms according to an aspect of the present invention;

FIG. 6a shows a schematic view of an exemplary standalone connector arm according to an aspect of the present invention;

FIG. 7b shows a schematic view of an exemplary standalone connector arm according to an aspect of the present invention;

FIG. 7a shows a schematic view of one tubular member of a pair of tubular members with a means to connect together the first tubular member and the second tubular member according to an aspect of the present invention;

FIG. 7b shows a schematic view of a pair of joined tubular members according to an aspect of the present invention;

FIG. 8a shows a schematic view of a pair of tubular members with a means to connect together the first tubular member and the second tubular member according to an aspect of the present invention;

FIG. 8b shows a schematic view of a pair of joined tubular members according to an aspect of the present invention;

FIGS. 9a, 9b, 9c, 9d show schematic views of exemplary configurations of proximal openings of a tubular member ac cording to an aspect of the present invention;

FIGS. 10a, 10b, 10c show schematic views of an exemplary device including a pair of tubular members with elliptical proximal openings according to an aspect of the present invention;

FIG. 11 shows a schematic view of at least one biological structure inserted in two unconnected tubular members of an exemplary connector device according to an aspect of the present invention;

FIG. 12 shows a schematic view of two ends of a biological structure connected with an exemplary connector device according to an aspect of the present invention;

FIG. 13 shows a schematic view of an exemplary system of the present invention connecting a plurality of structures according to an aspect of the present invention;

FIG. 14a shows a schematic view of an exemplary device for use in a bypass procedure according to an aspect of the present invention;

FIG. 14b shows a schematic view of an opening in an artery according to an aspect of the present invention;

FIG. 14c shows a schematic view of an exemplary tubular member of a bypass device according, to an aspect of the present invention;

FIG. 15 shows a schematic view of one part of an exemplary bypass device connected to one opening of an artery and a second part of an exemplary bypass device connected to one end of a graft according to an aspect of the present invention;

FIG. 16 shows schematically an exemplary first bypass tubular member attached to one end of a graft and an exemplary corresponding second bypass tubular member attached to an opening in an artery, wherein the first bypass tubular member and the corresponding second bypass tubular member are connected together according to an aspect of the present invention;

FIG. 17 shows an exemplary system of the present invention used in a bypass procedure according to an aspect of the present invention;

FIG. 18 shows a flow chart of an exemplary method of using the device according to an aspect of the present invention;

FIG. 19 shows a flow chart of an exemplary method of using a system including a plurality of devices according to an aspect of the present invention;

FIG. 20 shows a flow chart of an exemplary method of using a system of the present invention in a bypass procedure according to an aspect of the present invention;

FIG. 21 shows a flow chart of an exemplary method of production of the device according to an aspect of the present invention;

FIG. 22 shows a floss chart of an exemplary method of production of connection means for connecting one tubular member to the other tubular member of a pair of tubular members according to an aspect of the present invention;

FIG. 23 shows a flow chart of an exemplary method of production of connection means for connecting one tubular member to the other tubular member of a pair of tubular members according to an aspect of the present invention; and

FIG. 24 shows a flow chart of an exemplary method of production of connection means for connecting one tubular member to the other tubular member of a pair of tubular members according to an aspect of the present invention.

### DETAILED DESCRIPTION

In one aspect the present invention is of a connecting device. The connecting device may be a device for joining body tissues, wherein a body tissue may be inserted into the device or part thereof The device may be for joining tissues in humans, mammals and animals. The device may be for joining biological tissues found internally. In an additional aspect the present invention is of a connecting arm for connecting multipart anastomosis devices. In a further aspect the present invention provides a method of using a connecting device. The present invention provides uses of such a device, for anastomosis, surgery, bypass, wound treatment and a combination thereof. In a still further aspect the present invention is of a method of production of the connecting device. The present invention provides a system for replacing and/or augmenting suturing in a bypass procedure, wherein the system may include a plurality of connector devices of the present invention. It is envisioned that the present invention may be used in the joining of any suitable tissues and body structures in any suitable area of the body.

The devices of the present invention provide uniform and optimal joining of the exposed ends/openings of the biological structures and/or grafts to be connected. The devices do not result in holes at the join of the inserted biological structures, such as those resulting from the use of sutures. As such, there is less leaking than in suturing. Furthermore, the devices of the present invention hold the biological structures firmly at all stages of the connection. Moreover, the devices of the present invention are configured to expand and contract according, to the dimensions of the inserted structure and thereto re do not exert substantially any external pressure on the biological structure. Due to the devices not exerting pressure on the biological structure/s, the devices may prevent pressure sores and related infection. Further, the lack of pressure by the devices may avoid causing narrowing of the joined biological structures. The devices of the present invention are relatively facile to use and facilitate a method of joining biological tissues/s, which is fast and less time consuming than the commonly used method of suturing.

The terms ‘first’ and ‘second’ with respect to the tubular members and devices are used arbitrarily and are meant for descriptive purposes to distinguish between each of the pair of tubular members or devices. As such, a first tubular member or a first device can be described as the second tubular member or second device respectively and vice versa as long as there is consistency and, the same term is used to describe that tubular member or device in a particular embodiment.

As used herein the term ‘arm’ may include any suitable component, which projects or can project from and over a tubular member and extend longitudinally with respect to the tubular member and is sized to connect to a second corresponding tubular member at any suitable position of the second corresponding tubular member. The arm may be a relatively narrow projection with respect to the diameter of the tubular member. The arm may be configured so that there is a tension in the arm, which may result in a return to the original diameter or length or position of the arm when the arm is pulled or pushed or expanded or a combination thereof.

As used herein the term ‘arch’ may include any suitable arcuate configuration of a connecting arm, which can be manipulated by pulling to provide a larger diameter and which will return to the original diameter when the pulling force is removed.

As used herein the term ‘connecting arm’ may include any suitable arm, which includes suitable attachment members or connecting means at a free end of the connecting arm for connecting a tubular member to an unconnected tubular member and facilitating a unitary device. The terms ‘connecting arm’, ‘moveable arm’, ‘arcuate arm’ ma be used interchangeably.

As used herein the term ‘prong’ when referring to the prong of an arm and the attachment means of the arm, may include any suitable projection, which may be pointed or not pointed and which may be configured to facilitate hooking of the connecting arm,

As used herein the terms ‘eversion’, ‘evert’, ‘everted’, ‘everting’ refers to the turning inside out of an end/opening of a structure, such as a biological structure or a synthetic graft, so that the inside layer is exposed. The eversion may be performed over a surface such as the opening or an edge of the opening or part thereof of the device of the present invention.

As used herein the term ‘tubular braid’ may include any suitable configuration with properties similar to a Chinese finger trap, wherein the configuration reversibly facilitates radial enlargement when the extremities are pushed inwards and reversibly facilitates radial contraction when the extremities are pulled outwards and/or pushing forces are removed. The term includes any tubular braid with suitable dimensions and suitably configured for use on a biological tissue. The term ‘tubular braid’, ‘braided sleeve’, ‘tubular plait’, ‘braid’ and ‘plaited sleeve’ may be used interchangeably.

As used herein the term ° radial enlargement/expansion may include, but is not limited to an increase of the radius and/or diameter of a tubular structure or similar shaped structure without external addition or deletion to the original structure.

As used herein the term ‘reversible radial enlargement/expansion’ may include, but is not limited to a return of the size of the radius/diameter towards and/or to its original radius/diameter on removing the forces facilitating the radial enlargement. The term may include a return to a radial size which is a reduction of the radial enlargement, but which may not be the same size as the original radius.

As used herein the term ‘radial contraction’ may include, but is not limited to a decrease of the radius and/or diameter of a tubular structure or similar shaped structure without external addition or deletion to the original structure.

As used herein the term ‘reversible radial contraction’ may include, but is not limited to a return of the size of the radius towards and/or to its original radius on removing the forces facilitating the radial contraction. The term may include a return to a radial size which is an increase of the radial contraction, but which may not be the same size as the original radius.

As used herein the term ‘joining’ may include, but is not limited to contacting and connecting in any suitable way in order that a plurality of structures or parts thereof are attached. The term may include connecting the two parts of a biological structure resulting from severing or cutting of the original uncut biological structure, such that the joining will reform the original uncut biological structure. The term may include connecting two previously unconnected structures.

As used herein the term ‘biological structure’ may include, but is not limited to any suitable biological tissue, which can be accommodated in the device of the present invention. The two sides of a cut biological structure may be referred to in some examples as two separate biological structures.

As used herein the term ‘pulling’ may include, but is not limited to a tugging force applied to the connector arm extension which may result in an increase in the diameter of an arched arm.

As used herein the term ‘attachment means’ may include, but is not limited to any suitable element or combination of elements, which facilitate connection of two separate parts.

The principles and operation of a device, such as a connector according to the present invention may be better understood with reference to the figures. The figures show non-limiting aspects of the present invention. The figures are not drawn to scale and the components and parts may not be drawn in the correct ratio.

FIG. 1 shows a schematic view of a device 10 according to an aspect of the present invention. In one aspect the device 10 may include a plurality of tubular members 12, 14. The device may include two tubular members 12, 14 as shown in FIG. 1. The device may include a pair of corresponding tubular members 12, 14. The tubular members 12, 14 may include a central cavity 16 and each tubular member 12, 14 may be configured to accommodate in the cavity 16 a structure, such as a biological structure or a synthetic structure, which is to be joined using the device 10 of the present invention. In one non-limiting example of a side to end connection or a side to side connection, at least one of the pair of corresponding tubular members 12, 14 may be configured to be disposed on a biological structure or a synthetic structure without the biological or synthetic structure being inserted through the tubular member 12, 14. The structure to be joined may be a tubular structure. The tubular member 12, 14 may be flexible. The tubular member 12 may include an elongated body 18, which may surround the central cavity 16 and which may include an opening 20 at a first proximal extremity 22 and an opening 24 at a second distal extremity 26. In some embodiments, the opening 24 at the distal extremity 26 may be defined as the opening through which a structure to be joined may be inserted and the opening 20 at the proximal extremity 22 may be defined as the opening where the end of the structure to be joined is positioned. In an example, wherein a tubular member is disposed on a biological structure or synthetic structure, without the structure hung inserted through both openings of the tubular member, the distal extremity may be defined as the end which directly sits on the structure. The end of the structure to be joined may be, but is not limited to a cut end, a severed end, a damaged end and an extremity. The tubular member 12, 14 may be an elongated structure 18 with a cavity 16, which may be substantially hollow extending longitudinally 28 between the first opening 20 and the second opening 24. The dimensions of the tubular member 12, 14 and the internal cavity 16 therein may be constructed to optimally accommodate an inserted structure. In some embodiments, different sizes of tubular members 12, 14 may be made for using with different types and sizes of biological structures and synthetic structures, such as synthetic grafts.

The length 28 of the tubular member 12, 14 of the present invention may be adjusted according to the end use of the device 10 and according to the type and length of the biological vessel/s to be connected. The length 28 of the tubular member may be, determined so that the device 10 is long enough to hold the biological structure and/or synthetic structure. The length 28 may be calculated so that the device 10 will be as short as possible for easy insertion of the biological or non-biological structure within the device 10. In addition, in one non-limiting example the length 28 may be optimized for insertion of the device 10 into the body by endoscope. The length 28 of a tubular member 12, 14 may be any suitable length 28 and may be a length 28 of from about 3 mm. In one non-limiting example the tubular member 12, 14 may have a length 28 of up to about 12 mm. The diameter 30 of the tubular member 12, 14 may be any suitable diameter 30. In some non-limiting examples the diameter 30 of the tubular member 12, 14 may be from about 2 mm. The size of the diameter 30 of the tubular member 12, 14 may depend on the size of the diameter of the biological structure and/or non-biological structure to be joined.

The diameter 30 of the tubular members 12, 14 may be changeable, such that a tubular member 12, 14 is configured to be reversibly radially expandable. In such a way a tubular member 12, 14 may expand to accommodate a larger diameter of an inserted structure and may revert to about the original dimensions with contraction of the inserted structure. The tubular members 12, 14 of the present invention may be formed with a plurality of different structures and may use different methods for facilitating the reversible radial expansion. The different structures may use a combination of methods for facilitating reversible radial expansion. The type of configuration used to construct the device 10 may include any suitable construction, which will facilitate such a substantially reversible change in size. The tubular member 12, 14 may have a set put into it so that it is initially open with a larger diameter 30 to aid in insertion of a structure, such as a biological structure into the tubular member 12, 14.

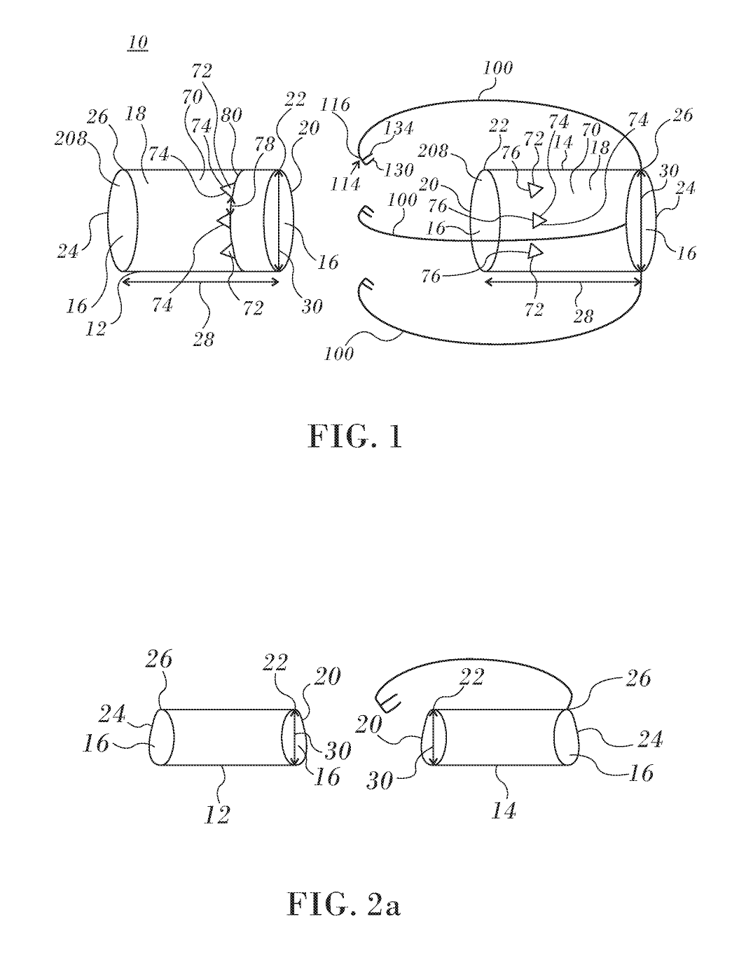

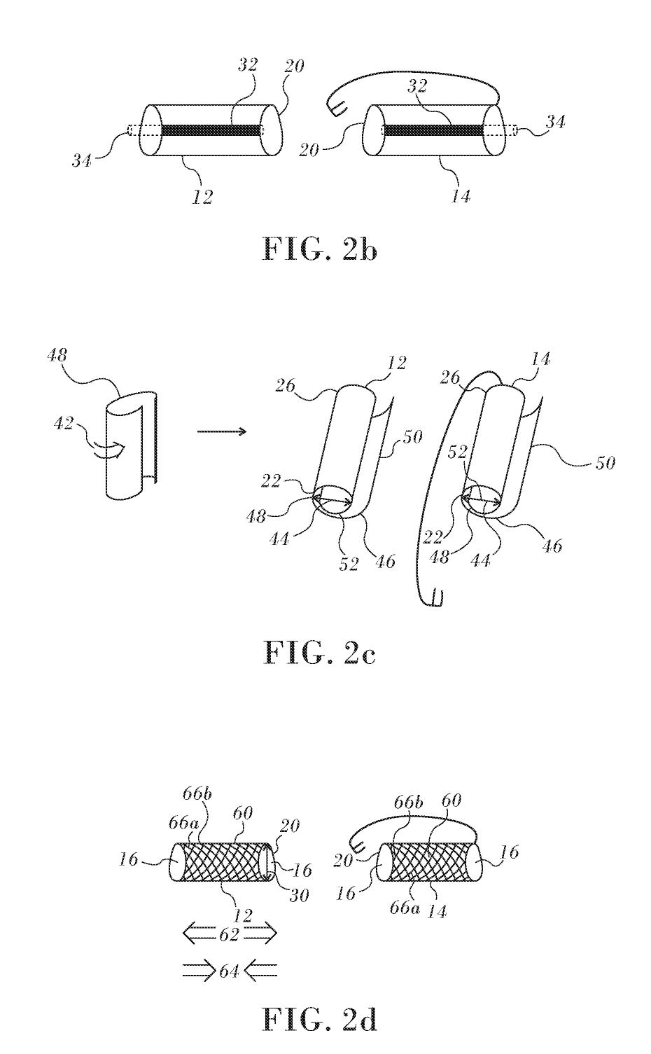

The device 10 may include tubular members 12, 14, which ate constructed from completely formed tubular members as shown schematically in FIG. 1 and FIG. 2a. Complete tubular members may be configured as complete rings. Complete tubular members may not include a split in the body 18 and the tubular member completely encircles and surrounds the cavity 16 of the tubular member 12, 14. A complete tubular member may be configured for reversible radial expansion using the properties of the material the tubular member 12, 14 is constructed from. The tubular member 12, 14 may be constructed from a material which can reversibly expand according to a change in diameter of an inserted structure. The tubular member 12, 14 may be made from a flexible material. In some embodiments, the tubular member 12, 14 may be made of a shape-memory material, such as, but not limited to nitinol. A tubular member 12, 14 which is made from a shape-memory material may be designed with an original relatively rigid configuration such that the diameter 30 is of suitable dimensions for optimal fit of a biological structures to be treated. Before use, the tubular member 12, 14 may be cooled to a temperature below the critical temperature of the shape-memory material. At such a temperature, the alloy is flexible and can be bent easily into any suitable shape for easy insertion of the biological and/or non-biological structures into the tubular member 12, 14. When the tubular member 12, 14 warms up to body temperature, the shape-memory material is heated to its critical temperature and transforms into its original more rigid arrangement. As such, using a change in temperature, the tubular members 12 14 and the device 10 may be configured for reversible radial enlargement and reversible radial contraction.

In some embodiments, the tubular members 12, 14 may be constructed from tubular members, which include a split 32 as shown schematically in FIG. 2b. The tubular members 12, 14 may be split longitudinally. The tubular members 12, 14 may be constructed from a length of material configured in a tubular, ringed conformation with a longitudinal split. The split 32 may facilitate accommodation of the changing dimensions of a biological structure or synthetic structure. The dimensions may change according to the flow of blood. The tubular member 12, 14 with a split 32 may expand and contract by opening of the split 32 to increase the gap 34. In some embodiments, the gap 34 may be small, such as less than about 10 microns. The size of the gap 34 of the split 32 may increase with increasing diameter of an inserted structure and the size of the gap 34 may decrease when the inserted structure contracts. A tubular member 12, 14 with a longitudinal split 32 may not be restricted by the constraints of the internal dimensions of a complete and unsplit tubular member. In addition, a tubular member 12, 14 with a split 32 may be configured for reversible radial expansion using the properties of the material the tubular member 12, 14 is constructed from. The tubular member 12, 14 may be constructed from a material which can reversibly expand according to a change in diameter of an inserted structure, such as but not limited to a shape-memory material.

In some embodiments, the tubular member 12, 14 may be constructed from a furled or curled up member as shown schematically in FIG. 2c. The furled member may be constructed from a flexible material. The furled member may be constructed from a material wherein the flexibility is sufficient to provide a tension in the resulting shaped member. In such an embodiment, a length of material 40 may be longitudinally furled 42 to result in a formed inner tubular cavity 44, which is completely or partially surrounded by at least one additional layer of the length of material 46. One longitudinal side 48 of the curled member is configured as part of a surface of the inner cavity 44 and the second longitudinal side 50 is configured as an additional layer 46 exterior to the inner cavity 44 and surrounding the inner cavity 44 and is not configured as part of the inner cavity 44. The dimensions of the inner cavity 44 are not restricted as in the complete and unsplit tubular member of FIG. 2a. The length of tubular member may be furled to facilitate a reversibly expandable central cavity of the tubular member, wherein the central cavity does not include a gap. The dimensions of the inner cavity 44 may change according to the dimensions of the inserted structure. A structure, such as, but not limited to a blood vessel, which is inserted into the tubular member 12 may have contracted dimensions when it is initially inserted into the tubular member 12. The tubular member 12 may be configured to hold the contracted structure sufficiently tightly. When the blood flow increases, the inserted structure may expand and the pressure of the increased diameter of the inserted structure on the wall of the tubular cavity 44 may cause the length of material 40 to unfurl to a degree in order to resize to a tubular member cavity 44 with a larger diameter 52 according to the larger diameter of the inserted structure. When the inserted structure has less blood flowing and contracts and the pressure on the sides of the tubular member cavity is removed, the tension in the furled material may allow refurling and returning of the inner cavity 44 to the original smaller dimensions. A furled tubular member may include any suitable number of furls rolled over each other. FIG. 2c shows one furl, wherein the number of furls as defined herein may be the number of layers which completely cover the inner cavity. In addition, in some embodiments the furled member may be constructed from a material, such as a shape-memory material, which can adapt to a change in radial dimensions of an inserted structure in the cavity. The furled member may facilitate a reversible radial expansion using a combination of the properties of the material and the properties of the furl described hereinabove. A furled tubular member provides a tubular member, which fully encircles without any gaps, a structure inserted in its cavity. The furled tubular member may be constructed, such that the cavity is a complete tubular cavity without a gap when it is in both an expanded state and a contracted state.

In some embodiments, the tubular member 12, 14 may be constructed from a braided tubular member 60 as shown schematically in FIG. 2d according to an aspect of the present invention. The braid 60 may provide a tubular member 12, 14 with reversible radial sizing to accommodate different and changing dimensions of an inserted structure. The diameter 30 of the cavity 16 of a braided tubular member may change according to the pulling 62 and pushing 64 forces applied on it, such as resulting, from an expanded or contracted structure inserted in the cavity 16. The braid 60 may be pushed inwards 64 to provide a tubular member, 12, 14 with a cavity 16 with a larger diameter 30 and the braid 60 may be pulled outwards 62 to provide a tubular member 12, 14 with a cavity 16 with a smaller diameter 30. The braid 60 may be similar to the tubular braid of a Chinese linger cuff. The braid 60 may be constructed from a plurality of filaments or lengths, such as 66a, 66b, which are braided or interwoven to facilitate reversible radial enlargement of the tubular member when a pushing force is applied to at least one end of the tubular member and reversible radial contraction when a pulling force is applied to at least one end of the tubular member. The braid 60 may be as described in US Patent Application Publication No. 20150351768 to the same inventors, which is incorporated as reference herein in its entirety. In addition, a braided tubular member may be configured for reversible radial expansion using the properties of the material, the braided tubular member is constructed from. The braided tubular member may be constructed from a material which can reversibly expand according to a change in diameter of an inserted structure, such as but not limited to a shape-memory material.

In one non-limiting example at least one part of the device may be constructed with a net type configuration. Such a configuration may facilitate greater flexibility of the device. A device, which includes a net or lattice may be configured so that the proximal extremities of the parts of the device in contact and in close proximity to the end/s of a biological structure to be joined are not netted in order to prevent leaking and to provide an even edge for eversion of the biological structures.

Referring back to FIG. 1, the device 10 may include an external surface 70. A structure inserted into a tubular member 12, 14 may be everted over the edge of the proximal opening 20. The proximal opening 20 may be configured to provide an even edge for eversion. An even edge may provide an even surface of the cut structure to be joined for optimal joining. Eversion may provide an exposed internal part of the end of the inserted structure, which may be fixed to the external surface 70 of the tubular member 12, 14 with a means 72 to hold and fix an inserted biological structure or synthetic graft to the tubular member 12, 14. In some embodiments, the means 72 may include a plurality of protrusions 72, which protrude out from the external surface 70 of the tubular members 12, 14. The plurality of protrusions 72 may be configured to pierce the tissue of an everted part of the inserted structure and transfix it to the tubular member 12, 14. Non-limiting examples of suitable protrusions 72 include spikes, prongs, hooks and pointed extensions. The protrusions 72 may be applied to the external surface 70 of the tubular members 12, 14. In some embodiments, the protrusions 72 may be constructed from a surface of the tubular member 12, 14, such as by punching out a spike from the body 18 of the tubular member 12, 14 formed from a partial perforation or partial cutout of the tubular member 12, 14. The perforation may be a superficial perforation or a perforation all the way through the surface of the tubular member 12, 14. In one example, the cutout may be two sides 74 of a triangular or pointed spike shape, such that the base 76 of the triangle is formed from the tubular member surface 70 and the sides 74 of the pointed spike can freely protrude from the fixed base 76 of the tubular member 12, 14. In an alternative example, which is not shown in the figure, the cutout may be a single straight or angled cut. The spike 72 may be cut out by any suitable technique, such as but not limited to using a laser or puncher. A stitch or equivalent may be introduced about the perforation or cutout in order to maintain the dimensions of the spike 72. The size of the protrusions 72 may be any suitable dimensions that facilitate transfixing the structure inserted in the device 10. The size of the protrusion 72 may also take into account the internal environment of the body and be sized so as not to contact any other biological structures in the body in the vicinity of the site of the anastomosis or other type of joining. The plurality of protrusions 72 may be equidistant from each other and may be spaced apart along the circumference of the body 18 of the tubular member 12, 14. The tubular member 12, 14 may include a plurality of protrusions 72, which may be any suitable number of protrusions 72. In some embodiments, the number of protrusions 72 may be about 5 or more. The protrusions 72 may be positioned an equal distance from the proximal opening 20 and along one line 80 of the circumference of the tubular member 12, 14. The protrusions 72 may be positioned along more than one line of the circumference of the tubular member 12, 14. The protrusions 72 may be positioned near to the proximal opening 20 for optimal fixing of the inserted structure to the device 10, and facilitating an exposed open end of the inserted structure positioned at the proximal opening 20 of the tubular member. The nearer the protrusions 72 to the proximal opening, 20, the easier it may be for a user to evert the inserted structure and fix it with the protrusions 72. The plurality of protrusions 72 may protrude outwards away from an exterior surface of the tubular member 12, 14 and may be pointing in a direction away from the proximal opening 20 towards the distal extremity 26 of the tubular member 12, 14. In one non-limiting example the base 76 of a spike 72 measures about 1.5 mm and the height of a spike 72 measured from the base to the peak is about 2 mm.

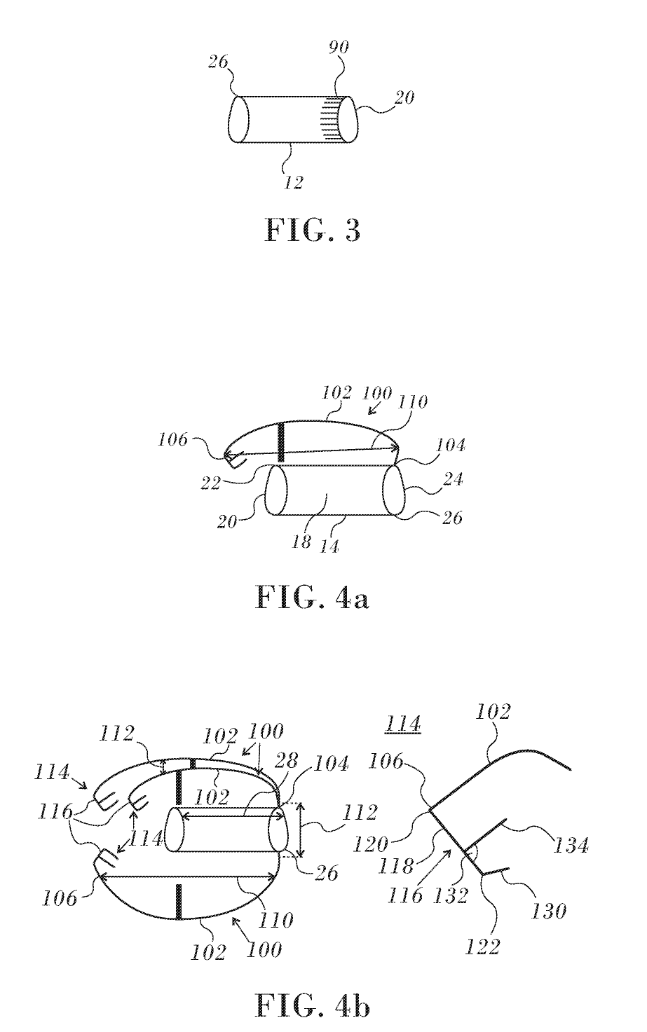

In one non-limiting example the means 72 to hold and fix an inserted structure may include a plurality of prongs 90. FIG. 3 shows a schematic view of an exemplary tubular member 12 with protrusions, which are prongs 90 adjacent to the proximal opening 20 according to an aspect of the present invention. As shown in FIG. 3, a plurality of prongs 90 are attached to the proximal opening 20 of the tubular member 12 and are positioned with the length, and free end of the prong protruding away from the proximal opening 20 towards the distal end 26 of the tubular member 12. A second corresponding tubular member is not shown in the figure, but similar prongs 90 may be included in a second corresponding tubular member, or any number of tubular members of a device of the present invention.

The device of the present invention may include a means to connect together a pair of tubular members, which may include a first tubular member 12 and a second tubular member 14. The device may include a pair of tubular members, wherein One of the pair of tubular members may include a means to connect the two tubular members together. Connection of the one tubular member to the corresponding tubular member may result in a unitary device. Connection may result in a linear unitary device. FIG. 1 shows an exemplary means 100 to connect the first tubular member to the second tubular member. The tubular member connecting means 100 may include at least one connecting arm 100 for connecting the first tubular member 12 to the second corresponding tubular member 14. In some embodiments, the tubular member 12, 14 may include a plurality of spaced apart extended arms 100. The tubular member 12, 14 may include any suitable number of connecting arms 100 to facilitate a stable and strong attachment of one of a pair of tubular members 12 (a first tubular member) to the corresponding tubular member 14 (a second tubular member) for optimal joining of biological and/or non-biological structures inserted therein. In some embodiments as shown in FIG. 1, the means 100 to connect the first tubular member to the second tubular member may include three connecting arms 100. The at least one connecting arm 100 may be formed with or fixedly attached to at least one of the tubular members 12, 14. In some embodiments only one tubular member 14 of a pair of tubular members 12, 14 may include the means to connect a pair of tubular members 12, 14. In a device 10 which includes a pair of tubular members 12, 14, such as a first tubular member 12 and a second tubular member 14, one of the tubular members may not include the at least one connecting arm 100, which is shown in FIG. 1 as the first tubular member 12 and the other tubular member of the pair of tubular members may include the at least one connecting arm 100, which is shown in FIG. 1 is as the second tubular member 14. The first tubular member 12 may, be configured for receipt and attachment of the second tubular member's 14 at least one connecting arm 100. The first tubular member 12 may include at least one means Corresponding to the attachment means on the free extremity of the second tubular member's 14 at least one connecting arms 100.

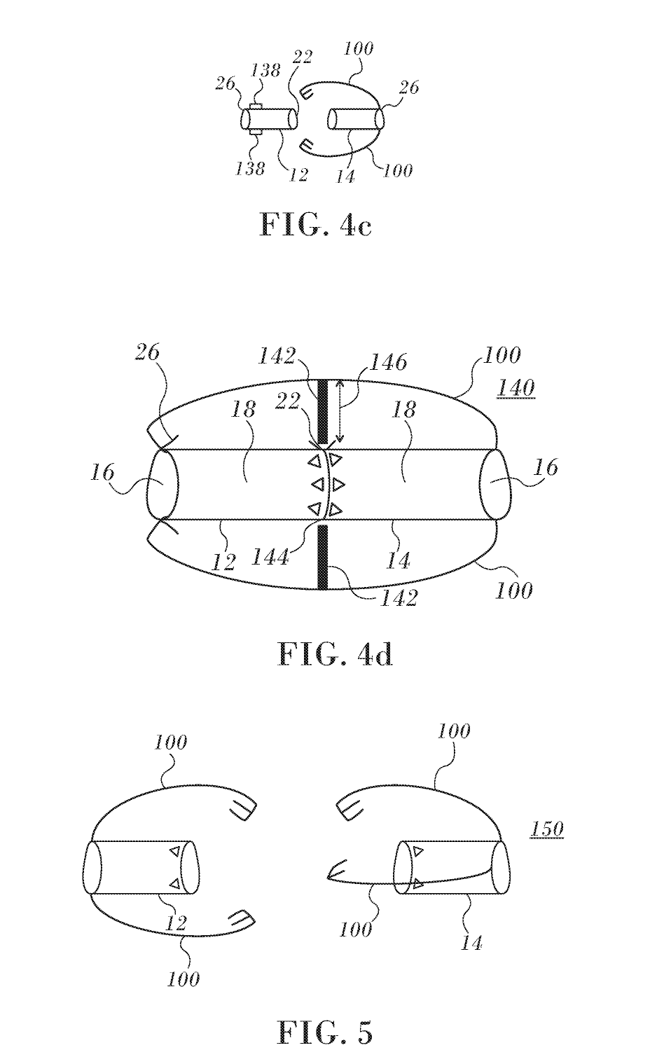

Referring to FIG. 4a which shows schematically an exemplary tubular member 14 with a connected at least one arm 100 according to an aspect of the present invention. The at least one extended arm 100 may include an extended length 102, the extension 102 including a first end 104 and a second end 106. The at least one arm 100 may be freely attached to the tubular member 14 by attachment of the first end 104 of the length 102 of the at least one arm 100 to an attachment point on the tubular member 14. Attachment may be adjacent to the distal extremity 26 of the tubular member 14, such that the extension 102 and the second end 106 of the extension 102 of the at least one arm 100 are restrictedly moveable about the attached first end 104 at the attachment point 104. In one non-limiting example, the at least one arm 100 may be attached at a suitable position on the body of the tubular member 14, which is not adjacent to the distal extremity of the tubular member 14. The at least one arm 100 may be fixedly attached to the tubular member 14. In some embodiments, the at least one arm 100 may be indirectly attached to the tubular member 14. A non-limiting example of an indirect attachment means may include a base or a protrusion or an additional extension of the at least one arm 100 to which the at least One arm may be attached. In one embodiment the at least one arm 100 may be detachably attached to the tubular member 14. The at least one arm 100 may be attached only about the first end 104 of the arm extension 102, such that the length of the extension 102 and the second end 106 of the extension are configured to extend freely above the body 18 of the tubular member 14 in a direction towards the proximal end 22 of the tubular member 14. The at least one arm 100 may be attached such that it overhangs the proximal end 22 of the tubular member 14.

In some embodiments, the at least one arm 100 may be of any suitable shape, which can facilitate a non-fixed diameter 110 between the two ends 104, 106 of the arm 100. The at least one arm 100 may be configured into a shape, which provides tension to the at least one arm 100. In some embodiments the at least one connecting arm 100 may be an arcuate arm, such that the arm is arched as shown in FIG. 4a.

In an embodiment, wherein there is a plurality of arms 100 as shown schematically in FIG. 4b each arm 100 may be attached in spaced apart relation 112 about the distal end 26 of the tubular member 14. Each arm 100 may be attached spaced apart on the circumference of the tubular member 14 adjacent the distal extremity of the tubular member 14. In a non-limiting example wherein the means 100 include three arms 100, each arm 100 may be positioned about one hundred and twenty degrees from the other arm along the circumference of the tubular member 14. In a non-limiting example wherein the means 100 include four arms 100, each arm 100 may be positioned about ninety degrees from the other, arm along the circumference of the tubular member 14.

The means 100 to connect together, a pair of tubular members 12, 14, such as the at least one arty 100 may include at least one attachment member 114 adapted to attach the tubular member with the fixed at least one arm to the other tubular member of a pair of tubular members, which in the non-limiting example shown in FIG. 4b may be the second tubular member 14 to the first tubular member 12. The attachment member 114 may include at least one hook 116. The hook 116 may be positioned at the free second end 106 of the at least one arm 100. The hook 116 may feature at least one prong 116 positioned at an angle to the arch 102 to facilitate hooking the distal end 26 of the corresponding first tubular member part 12 of the device 10. The at least one prong 116 may include a length of prong 118 with one end 120 of the prong joined to the free end 106 of the arch 102 and with a second end 122 of the prong, which is free and unattached. The at least one prong 116 may be configured as an angled hook extension 116 of the arch 102. The at least one prong 116 may include at least one protrusion. The at least one protrusion may include a first at least one protrusion 130.

The first at least one protrusion 130 may be positioned at the free end 122 of the at least one prong 116 and may protrude at an angle 132 to the length of the prong, angled towards the first fixed end 104 of the arm 100, facilitating at least part of a hooking means and configured to hook the distal end 26 of the corresponding first tubular member part 12 of the device 10. The first at least one protrusion 130 may be configured to attach into the cavity 16 about the distal opening of the corresponding first tubular member 12 to detachably fix the moveable arm 100 to the corresponding other tubular member 12 of the device 100. The at least one protrusion may include a second at least one protrusion 134, protruding at an angle to the length of prong, angled towards the first fixed end of the arm 104, the second at least one protrusion 134 protruding from a position along the length of the prong extension 118, but which is not at the end of the prong 122 and is spaced apart from the first at least one protrusion 130. The second at least one protrusion 134 may be longer than the first at least one protrusion 130. The second at least one protrusion 134 may be configured to allow the first at least one protrusion 130 to attach into the cavity 16 adjacent the distal opening of the corresponding other tubular member part 12 of the device 10 and to prevent displacement of an additional part of the arm 102 from entering the cavity 16 of the corresponding other tubular member part 12 of the device 10, such as the first tubular member 12. The first at least one protrusion 130 and the second at least one protrusion 134 may be angled to facilitate optimal and strong attachment to the second part 12 of the device 10. In an embodiment wherein the at least one arm 100 includes an arch 102, the diameter of the arch 102 may be determined according to the position it is fixed on the second tubular member 14 and the position it is to be attached on the first tubular member 12. In an embodiment, wherein the at least one arm 100 is fixed about the distal extremity 26 of the second tubular member 14 and is to be attached to the distal extremity 26 of the first tubular member 12, the diameter of the arch 110 may be equal to at least the combined length of the first tubular member 28 and the length of the second tubular member 28. The arch 102 may have limited flexibility that can facilitate a diameter 110 equal to at least the combined length of the first tubular member 28 and the length of the second tubular member 28 and the length of everted structures protruding over the proximal extremity 22 of the first tubular member 12 and the second tubular member 14. The arm 100 may be adapted to attach to the second, corresponding tubular member 12 of the device 10 by the tension of the arch 102 at manipulating the tension of the arch 102. The arm 100 may be detached from the second corresponding tubular member 12 by a surgeon after attachment, for example in order to adjust the connection or remove the device.

As shown schematically in FIG. 1, in some embodiments one of the pair of tubular members, which does not include the fixed at least one arm attachment member, such as the first tubular member 12 may include at least one corresponding attachment means for attaching the at least one arm attachment member 100 The corresponding means may include an edge of the distal opening 24 of the first tubular member 12 on which the arm 100 can hook and a portion of the cavity 16 of the first tubular member 12 into which the hook 116 can attach. In some embodiments the cavity may include at least one slot into which the hook 116 or part thereof fits.

In an alternative embodiment, the corresponding attachment means may include at least one slot 138 in any suitable position on the tubular member without the fixed at least one arm and into Which the arm attachment member is configured to attach. The at least one slot 138 may be positioned near the distal end 26 of the tubular member 12 as shown schematically in FIG. 4c. The at least one slot 138 may be a unidirectional slot, such that the connecting arm 100 cannot detach.

FIG. 4d shops schematically an exemplary first tubular member 12 connected to a second tubular member 14 with a plurality of arms 100 according to an, aspect of the invention. The joining of the first tubular member 12 and the second tubular member 14 facilitates a unitary device 140. The first tubular member 12 and the second tubular member 14 are connected end to end, such that the proximal end 22 of the first tubular member 12 is in linear contact with the proximal end 22 of the second tubular member 12 and the plurality of tubular attachment means 100 fixed to the second tubular member 14 are detachably attached to the distal end 26 of the first tubular member 12. In order to prevent displacement of the optimally positioned tubular members 12, 14 with the proximal opening 22 of the first tubular member 12 and the proximal opening 22 of the second tubular member 14 adjacent to each other and linearly connected to provide a continuous unobstructed cavity 16 through the joined first tubular member 12 and the second tubular member 14, the device 140 may include elements 142 to prevent displacement of the first connected tubular member 12 and the second connected tubular member 12 of the unitary device 140. FIG. 4d shows schematically exemplary elements 142 to prevent displacement. As can be seen in FIG. 4d the at least one arm 100 may include at least one downward protruding member 142 adapted to prevent displacement, such as vertical displacement, horizontal displacement and angular displacement of at least one of a first tubular member 12 and a second tubular member 14 in a unitary connected device 140 in order to prevent the proximal ends of the tubular members 12, 14 moving away from each other and the resulting non-optimal contact of the ends of the structures to be joined. Vertical displacement may be defined as non-unitary displacement vertically up or down and horizontal displacement may be defined as non-unitary displacement to either side. Angular displacement may be defined as a combination of vertical and horizontal displacement. FIG. 4d shows each arm 100 with one downward protruding member 142. Each arm 100 may include any suitable number of displacement prevention means 142. The displacement prevention means 142 may be disposed at any suitable position of the arm 100. In some embodiments, the displacement prevention means 142 may be disposed adjacent to and in a vertical line with the joining line 144 of the two tubular members 12. 14. In one non-limiting example, the at least one arm 100 may include two displacement prevention means 142, one displacement prevention means 142 positioned to be adjacent to the proximal opening 20 of the first tubular member 12 after connection and a second displacement prevention means 142 positioned to be adjacent to the proximal opening 20 of the second tubular member 14 after connection. The displacement prevention means 142 may be sized so that the free extremity of the length 146 of the protruding member 142 contacts the body 18 of the tubular member 12, 14 when the tubular member 12, 14 is not displaced. In one non-limiting example at least one arm 100 of a plurality of arms 100 may not include a displacement prevention means 142.

FIG. 5 shows schematically an alternative embodiment of a device 150 which includes a pair of corresponding tubular members 12, 14, such as a first tubular member 12 and a second tubular member 12. In the non-limiting example the first tubular member 12 may include a connection means 100 to connect the first tubular member 12 to the second tubular member 14 and the second tubular member 14 may include a connection means 100 to connect the second tubular member 14 to the first tubular member 12. The first tubular member 12 may include at least one connecting arm 100 and the second tubular member 14 may include at least one connecting arm 100. The first tubular member 12 may include at least one corresponding means for attachment of the second tubular member's 14 at least one connecting arm 100 and the second tubular member 14 may include at least one corresponding means for attachment of the first tubular member's 12 at least one connecting arm 100. Each of the tubular members 12, 14 may include any suitable number of connecting arms 100 In one non-limiting example as shown in FIG. 5 each of the first tubular member 12 and the second tubular member 14 include two connecting arms 100. Although FIG. 5 shows the at least one connecting arms 100 as arcuate arms, this is not meant to be limiting and the at least one connecting arms 100 may be any suitable connecting arms 100 as described herein.

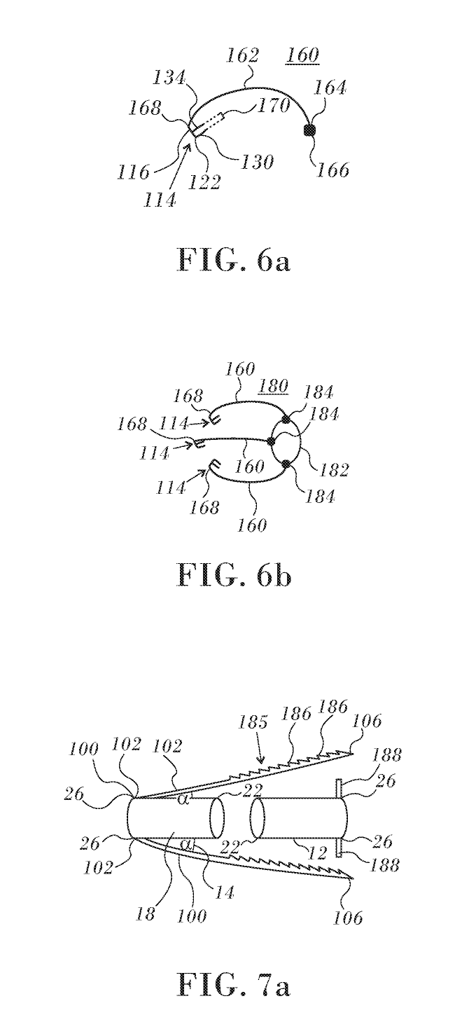

In one aspect, the present invention provides a standalone connecting arm. FIG. 6a and FIG. 6b show schematically exemplary standalone connecting arms 160 according to aspects of the present invention. The standalone connecting arm 160 may be for connecting a first part and a second part of a device for anastomosis. The connecting arm 160 may be for connecting two parts of a device wherein the device is for external application to at least one biological structure. The standalone connecting arm 160 may include an extended arch 162. The device may be a connector device of the present invention as described hereinabove. The device may be any other suitable device, which can employ the standalone connecting arm 160 to connect together a plurality of parts of the device. The device may be a device, for end to end anastomosis. The device may be a device, which is applied externally and which includes two unconnected parts, wherein each device part holds a structure to be joined, such as for example a blood vessel. A first part of the device may feature a first opening at a proximal end of the device, the first opening proximal to the anastomosis connection and an additional second opening at a distal end of the device, the second opening distal to the anastomosis connection. A second part of the device may feature a first opening at a proximal end of the device, the first opening proximal to the anastomosis connection and a second opening at a distal end of the device, the second opening distal to the anastomosis connection. The device may be a device, which includes corresponding attachment means for attaching a standalone connecting arm 160 and is configured such that a first end 164 of the standalone connecting arm 160 can attach with attachment means 166 to a first part of the device and the second end 168 of the standalone connecting arm 160 can attach with attachment means 114 to the second part of the device. Attachment means may be configured and used as described hereinabove for FIG. 4b. The connecting arm 160 may attach to any suitable position on the first device part and the second device part for holding the two parts of the device together and holding the two ends of an inserted structure together for treatment, such as end to end anastomosis. The connecting arm 160 may attach to a distal end or adjacent thereof of the first device part and the second device part.

FIG. 6a shows the standalone connecting arm 160 including an arm extension 162, the extension featuring a first end 164 and a second end 168. Each arm 160 of a plurality of arms may include attachment means 166 at the first end 164 and attachment means 114 at the second end 168. A standalone connecting arm 160 may include detachable attachment means 166 at the first end 164 to attach to one of the tubular members. Suitable attachment means 166 may include a clip, or a clamp for clamping to a distal end 26 (distal defined as distal to the exposed cut end of an inserted structure) of the first part of the device. facilitating a clamped arm restrictedly moveable about the clamped attachment. The attachment means 114 at the second end 168 of the standalone connecting arm 160 may include any suitable attachment means 114 to attach to the second part of a device. The attachment means 114 at the second end 168 of the standalone connecting arm 160 may include attachment means 114 as described hereinabove for FIG. 1 and FIGS. 4a-4d. The attachment means 114 may include at least one hook or prong 116 positioned at an angle to the arch 162 to facilitate hooking the distal end 26 of the second part of the device for attachment and production of a unitary device. The at least one prong 116 may include an angled extended length integrally formed at one end 168 of the arch 162, the at least one prong 116 including a second free end 122. The at least one prong 116 may include at least one protrusion, wherein the at least one protrusion may feature a first at least one protrusion 130 positioned at the free end 122 of the prong 116 and protruding at an angle towards the first end 164 of the arm 160. The first at least one protrusion 130 may be configured to hook the distal end 26 of the second part of the device and to attach into the cavity 16 of the second part of the device to detachably fix the moveable arm 160 to the second part of the device. The at least one protrusion may include a second at least one protrusion 134, which may be fixed along the length of the prong and may protrude at an angle towards the first end 164 of the arm 160 the second at least one protrusion 134 spaced apart from the first at least one protrusion 130 and not at the end 122 of the prong. The second at least one protrusion 134 and the gap 170 between the first at least one protrusion 130 and the second at least one protrusion 134 may be sized and configured to only allow the first at least one protrusion 130 to attach into the cavity 16 of the second part of the device and to prevent an additional part of the arm 160 from entering into the cavity 16 of the second part of the device. The second at least one protrusion 134 may be longer than the first at least one protrusion 130. The standalone connecting arm 160 may include displacement prevention means as described hereinabove.

FIG. 6b shows an exemplary standalone connecting means 180, which features a plurality of connecting arms 160 according to an aspect of the present invention. A plurality of connecting arms 160 may be attached at one extremity onto a ring 182, wherein the ring 182 is sized to attach to a tubular connector member. A plurality of attachment clips 184 may be disposed spaced apart on the ring 182. The number of attachment clips 184 may be determined for stable and secure attachment of the standalone connecting means 180 to a tubular member of a device. The clips 184 may be used to clip the ring 182 about the distal opening 24 of one part of the device. The plurality of connecting arms 160 may include attachment means 114 at a second extremity 168 of the connecting arms 160. for attaching one part of the device to the second part of the device. The remaining part/s of the connecting arm/s 160 and the attachment means 114 at the second end 168 of the plurality of connecting arms 160 for connecting the first part of the device to the second part of the device may be as described hereinabove for FIG. 1. FIGS. 4a-4d and FIG. 6a.

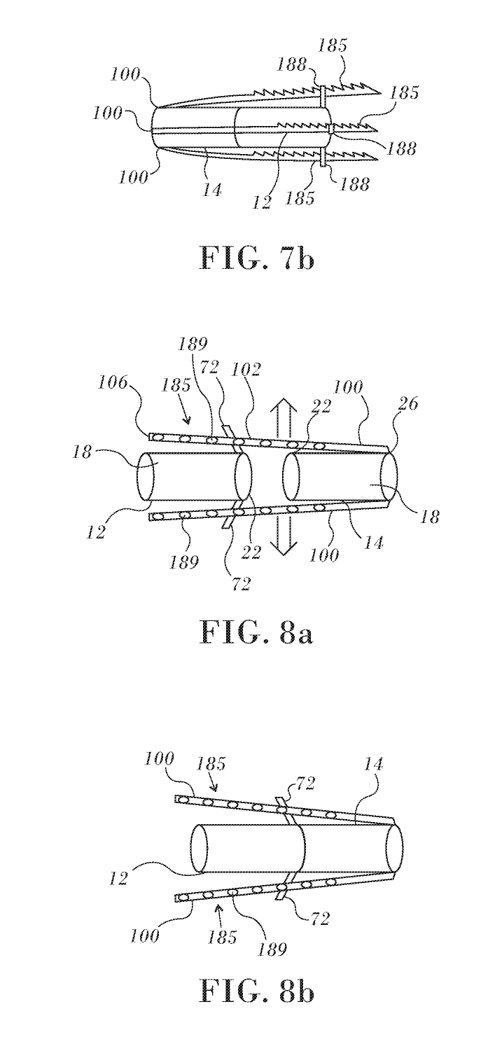

The present invention provides an additional example of a means 100 to connect together the first tubular member and the second tubular member of a pair of tubular members as shown schematically in FIG. 7a. The means to connect together two tubular members may include at least one arm 100. The at least one arm 100 may be attached at the first end of the arm extension 102 to a suitable position on one tubular member 14 of the pair of tubular members 12, 14. A suitable position on one tubular member ma be about the distal extremity 26 of the tubular member 14. In one non-limiting example a suitable position may be at another position on the tubular member 14 and the at least one arm 100 may be sized and angled according to its position. The at least one arm is attached, such that the length of the extension 102 and the second end 106 of the extension are configured to extend freely away from the body 18 of the tubular member 14 in a direction towards the proximal end 22 of the tubular member 14. The at least one arm 100 may be attached such that it overhangs the proximal end 22 of the tubular member 14, and is sized in order to be capable of attaching to a corresponding connection means on the other tubular member 12 without the fixed at least one arm 100. The at least one arm 100 may be a linear arm featuring a linear arm extension. A plurality of at least one arms 100 may be attached in spaced apart relation to each other along the circumference of the tubular member 14. The plurality of arms 100 may be fixed to the tubular member 14 to facilitate the plurality of arms inclined at an angle away from the body 18 of the tubular member 14 in a direction towards the proximal end of the tubular member 14. The plurality of arms 100 may extend pass the proximal end and may overhang the proximal end of the tubular member 14. The at least one arm 100 may include at least one attachment member 185 tot connecting to the corresponding tubular member 12 without the fixed at least one arm 100. The at least one attachment member 185 may include a ratchet type means 185, such as but not limited to a ratchet tie means 185, which may be disposed along the length of the arm extension. The ratchet type means 185 may include a plurality of angled unidirectional teeth 186 or notches 186 spaced apart along the extension 102 of the arm. The corresponding connect on means on the other tubular member 12 without the fixed at least one arm 100, to which the at least one attachment member 185 is configured to attach to, may include at least one elevated slit 188. A plurality of the at least one slits 188 may be fixed to the other tubular member 12 to provide elevated at least one splits in spaced apart relation protruding from the other tubular member 12. The at least one slits 188 may be configured, such that the arm extension 102 with the ratchet type angled teeth 186 or notches 186 may be inserted there through and where the at least one slits 188 may include a unidirectional movement means to prevent the ratchet type means 185 from reverse movement out of the elevated slit 188. The elevated slits 188 may be positioned at any suitable position on the corresponding tubular member 12. In one non-limiting example the at least one elevated slit 188 may be disposed about the distal end 26 of the corresponding tubular member 12. The arm 100 may have limited flexibility. The arm extension 102 may be angled according to the elevation of the at least one slit 188 in order that insertion of the arm extension 102 through the slit 188 may create a tension in the arm 100, which facilitates the area 100 proceeding though the slit 188 as the two proximal ends 22 of the device are brought together by a user. The plurality of slits may be fixed in spaced relation on the body of the tubular member to which the plurality of arms is not fixed, to correspond to a respective arm of the plurality of arms on the corresponding tubular member. The at least one slit 188 may be positioned and elevated so that the arm 100 does not affect the joining of the biological and non-biological structures inserted in the two tubular member parts 12, 14 of the device. The at least one slit 188 may be positioned and elevated to a height below the height of the respective arm of the corresponding tubular member so that the elevated slit is lower than the corresponding arm 100 in order to create the desired tension. The plurality of elevated slits 188 are configured for receiving one of the plurality of arms 100 through the slit and for preventing the arm from reverse displacement out of the slit. The plurality of elevated slits are configured for promoting the arm to proceed through the slit until the proximal opening of the two corresponding tubular members 12, 14 or structures everted thereon are in contact. A user may insert each of the plurality of arm extensions 102 into the respective corresponding slit 188 and then a user may push the two tubular members 12, 14 together to contact the proximal ends 22 with each other. As the user pushes the two tubular members 12, 14 together, the arm extensions 102 will proceed further through the slit 188 until the tubular members 12, 14 are in contact and are no longer moved. Alternatively, a user may pull each of the arm extensions 102 of a plurality of arms 100 on a tubular member 14, through the corresponding slits 188 of a corresponding second tubular member 12, until contacting together the exposed ends of the structures to be joined, which may be fixed at each of the two proximal ends of the two parts of the device. The means 100 to connect together the first tubular member and the second tubular member of a pair of tubular member's 12, 14 is easy to use and a user can control the joining of the two parts of the device. FIG. 7b shows schematically a unitary device, wherein one tubular member 14 of a pair of tubular members 12, 14 includes at least one arm 100 with a ratchet type attachment member 185 and wherein each of the at least one arm 100 is connected to the corresponding unidirectional slit 188 of the second tubular member 12 of the pair of tubular members 12, 14.

The present invention provides a further example of a means 100 to connect together the first tubular member and the second tubular member of a pair of tubular members as shown schematically in FIG. 8a. The means to connect together two tubular members 12, 14 may include at least one arm 100, which may be attached at the first end of the arm extension 102 to one of the tubular members 14, such that the length of the extension 102 and the second end 106 of the extension are configured to extend freely away from the body 18 of the tubular member 14 in a direction towards the proximal end 22 of the tubular member 14. The at least one arm 100 may be attached to the distal end 26 of the tubular member 14 or to any other suitable position. The at least one arm 100 may be fixed to the tubular member 14 to facilitate the plurality of arms 100 inclined at an angle away from the body 18 of the tubular member 14. The at least one arm 100 may extend such that it overhangs the proximal end 22 of the tubular member 14. A plurality of at least one arms 100 may be positioned spaced apart about the circumference of the tubular member 14. In a non-limiting example wherein there are three arms 100, each arm may be about one hundred and twenty degrees from each other. In an example wherein there are four arms 100, each arm may be about ninety degrees from each other. The at least one arm 100 may be sized in order to be capable of attaching to a corresponding connection means on the other tubular member 12 of the pair of tubular members 12, 14. The at least one arm 100 may be sized and angled according to the position it is fixed on the tubular member 14 The at least one arm 100 may be a linear arm featuring a linear extension. The at least one arm 100 may include at least one attachment member 185 for connecting to the corresponding tubular member 12. The at least one attachment member 185 may include a plurality of holes 189 or openings 183 spaced apart along the length of the linear extension 102 of the arm. The corresponding connection means on the corresponding tubular member 12 to which the attachment member 185 is configured to attach to, may include at least one protrusion 72, such as a spike 72. The at least one protrusion 72 may be for fixing an everted structure to the tubular member 12, as described herein. The at least one protrusion 72 may also be configured to fit into and to be inserted into one of the openings 189 of the at least one attachment member 185 on the linear arm extension 102 to connect the pair of tubular members 12, 14. The at least one protrusion 72 may be angled to prevent reverse displacement out of the opening 189. The plurality of protrusions 72 may be spaced apart and fixed on the body 18 of the corresponding tubular member 12. The plurality of protrusions 72 may be positioned near the proximal end of the corresponding tubular member 12. In some embodiments, the at least one protrusion 72 for inserting into one of the holes 189 of the attachment member 185 may be disposed on the tubular member in a position such that the at least one protrusion 72 is not also used for fixing an everted structure. The at least one protrusion 72, such as a spike may be sized in order that it can be used for fixing a structure, such as a biological structure to a tubular member and have sufficient length to also be inserted into a hole 189 of the at least one attachment member 185 of the at least one arm 100. The plurality of protrusions 72 may be sized and fixed to the tubular member 12 without the fixed plurality of arms, such that the free ends of the plurality of protrusions 72 are positioned at an elevated height With respect to the corresponding arm 100 of the plurality of arms 100 fixed on the tubular member 14 with the fixed plurality of arms 100. The at least one arm 100 may be angled lower than the free end of the spike 72, so that the at least one arm 100 must be raised in order to insert the free end of the spike 72 though an opening 189 in the arm 100. The arm may have limited flexibility and the tension of the arm 100 resulting from the force to raise the relatively rigid arm 100 and the angle of the at least one protrusion 72 may prevent the at least one protrusion 72 from being displaced from a hole 189 after insertion. The two tubular members 12, 14 may be contacted together by a user and each of the at least one arms 100 may be raised in order that a protrusion 72 can be inserted into a suitable hole 189 in the arm 100 to facilitate a unitary device. FIG. 8b shows schematically a unitary device, wherein each of a plurality of spikes 72 of one tubular member 12 is inserted in a respective hole 189 of the at least one attachment member 185 of each of the at least one arms 100 of a second corresponding tubular member 14 to connect a pair of tubular members 12, 14 and form a unitary device.

Referring back to FIG. 1, the tubular members 12, 14 include a proximal opening 20 and a distal opening 24. The edges of the proximal opening 20 and the distal opening 24 may be smooth and even, proximal opening 20 and the distal opening 24 may be perpendicular to the length 28 of the tubular member 12, 14 as shown in FIG. 1. The entire proximal opening 20 may be perpendicular to the length 28 of the tubular member 12, 14. The resulting uniform perpendicular opening 20 provides a suitable, means for facilitating a straight edge over which to evert an end of a biological structure or other structure, so the open exposed end of the inserted structure is substantially perpendicular to the length 28 of the tubular member 12, 14. The perpendicular proximal opening 20 of the tubular member 12, 14 is configured so that structures to be joined are positioned end to end, wherein the two ends may be one hunched and eighty degrees to each other for straight end to end joining of the structures. The proximal opening 20 may be parallel to the distal opening 24. The proximal opening 20 and the distal opening 24 may be similarly sized, and shaped.

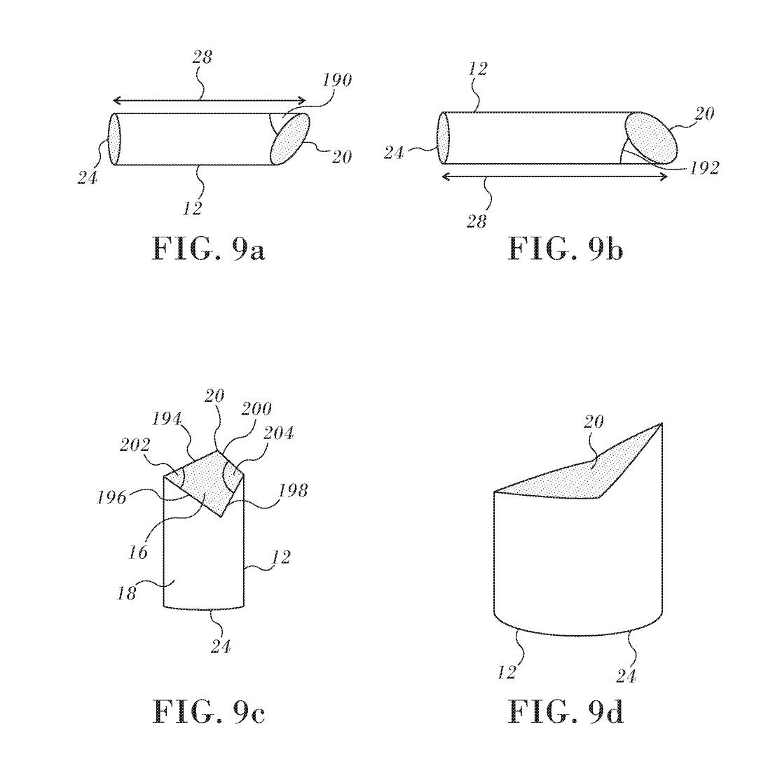

In some embodiments, the proximal opening 20 and/or the distal opening 24 or part thereof of the tubular member 12, 14 may not be perpendicular to the length 28 of the tubular member and/or may not be uniform. In some embodiments, the proximal opening 20 may be different from the distal opening 24. In some embodiments the proximal opening 20 may have different configurations according to the use of the device 10. The proximal opening 20 may provide the shape of the edge of an end or opening of a biological structure or non-biological structure to be joined. The shape of the proximal opening 20 may provide the angle of attachment of a biological structure or non-biological structure to be joined. In some examples and procedures the cut of the biological structure and/or non-biological structure may riot be a straight perpendicular cut and/or it may not be desirable for an everted edge of the biological structure and/or non-biological structure or part thereof to be perpendicular to the length 28 of the tubular member 12, 14. The proximal opening 20 may be constructed to provide the correct angle and/or shape for different types of joining and for connecting different shapes of openings and ends of biological structures and other structures. The proximal opening 20 may be any suitable shape, such as but not limited to rounded, elliptical, oval, straight edged, square, triangular, kite shaped, poly-sided and a combination thereof The proximal opening 20 may include a plurality of sides of a plurality of different lengths, which are at angles to each other. The shape of the proximal opening 20 may be carved from the body 18 of the tubular member 12, 14 to result in part of the tubular body 18 being an incomplete ring. FIGS. 9a-9d show schematically non-limiting examples of configurations of proximal openings 20 of a tubular member 12, 14 of the present invention according to an aspect of the present invention. FIG. 9a shows a circular proximal opening 20 of a tubular member 12, wherein the opening 20 is at an angle 190 to the length 28 of the tubular member 12, 9b shows a rounded proximal opening 20 at an angle 192 to the length 28 of the tubular member 12. FIG. 9c shows a proximal opening 20, wherein the opening 20 is a kite shaped quadrilateral and includes four sides 194, 196, 198, 200, wherein the first side 194 and the second side 196 of the opening 20 are of equal length and are joined at a first angle 202 to each other and the third side 198 and the fourth side 200 are of equal length, and are joined at a second angle 204 to each other. In the example shown in FIG. 9c the length of the first side 194 and the second side 196 are different from the length of the third side 198 and the fourth side 200 and the first angle 202 is different from the second angle 204. In the non-limiting example the side 196 and the side 198 are carved out of the body 18 of the tubular member 12. The surrounding part of the external body 18 of the tubular member 12, which includes the sides of the opening, sides 196 and 198 has a circumference with a cutout of the opening made by the sides 196 and 198. FIG. 9d shows a different schematic view of an exemplary tubular member 12 with a kite shaped opening 20. A tubular member 12 with an angled or non-uniform shaped proximal opening 20 may be suitable for use in a procedure, wherein the cut part to be joined of a structure has a shape, which is not circular or which is non-uniform, such as in a bypass procedure. A tubular member 12 with an angled or non-uniform shaped proximal opening 20 may be suitable for use in a procedure to provide certain predetermined angled joins, which are repeatable without the need for precise cuts of the biological structures and non-biological structures to be joined.

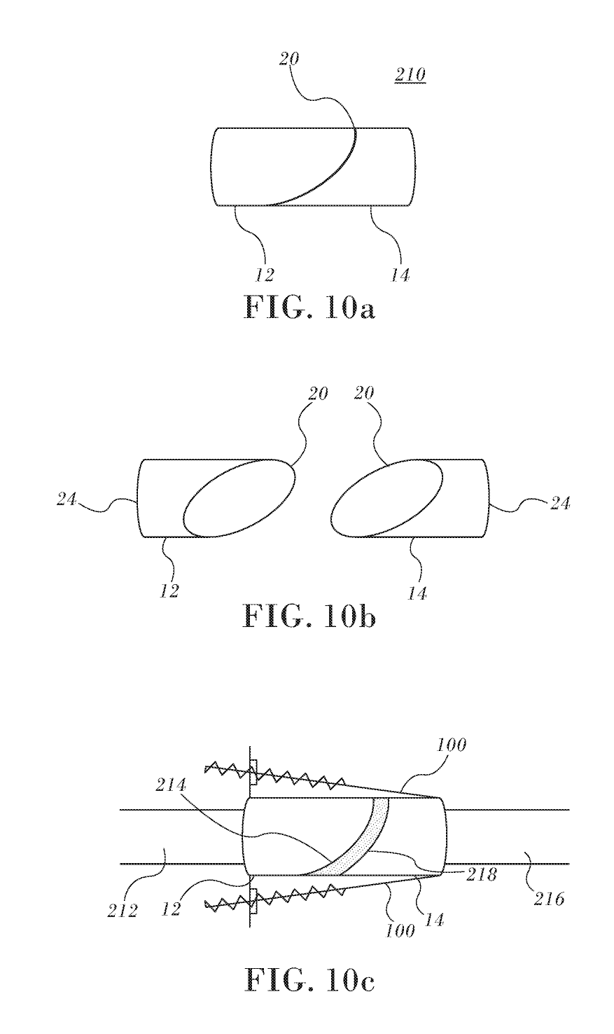

In some embodiments a tubular member 12, 14 with a proximal opening 20, which is angled, such as, but not limited to an elliptical proximal opening 20 may be suitable for use in an end to end connection. Such a tubular member may be advantageous as an angled extremity of a body vessel may provide more surface area and a larger area of contact for the joining of the structures inserted therein, which may be beneficial and may be easier for a surgeon to join. FIG. 10a shows schematically a device 210, which includes two corresponding tubular members 12, 14, wherein the proximal opening 20 of each of the two corresponding tubular members 12, 14 is elliptical, according to an aspect of the present invention. FIG. 10b shows the disconnected tubular members 12, 14, wherein the angled proximal opening 20 of one of the pair of tubular members 12 corresponds With the angled proximal opening 20 of the other tubular member 14 for joining of one tubular member's 12 proximal opening 20 to the other tubular member's 14 proximal opening 20 to provide a substantially linear cavity leading from one tubular 12 member to and through the second tubular member 14. In the example shown, the proximal opening 20 is not parallel to the distal opening 24 of the tubular members 12, 14. Tubular members 12, 14 with a proximal opening 20 which is elliptical or oval may provide a means for joining structures of different diameters. FIG. 10c shows schematically a pair of tubular members 12, 14 with structures to be joined inserted therein, wherein each of the tubular members includes an elliptical proximal opening 20, according to an aspect of the present invention. An end of a biological structure 212 inserted in the first tubular member 12 may be everted over the proximal opening 20 of the first tubular member 12. The angled tubular member opening 20 facilitates configuring the exposed open extremity 214 of the evened biological structure with the same angle and shape as the proximal opening 20 of the first tubular member 12. An end of a biological structure 216 inserted in the second corresponding tubular member 14 may be everted over the proximal opening 20 o the second tubular member 14. The angled tubular opening 20 facilitates configuring the exposed extremity 218 of the everted biological structure with the same angle and shape as the proximal opening 20 of the second tubular member 14, Connecting arms 100 as described herein may be used to connect the first tubular member 12 to the second tubular member 14 and keep them together in order to join the exposed ends 214, 218 of the biological structures 212, 216 inserted therein. FIG. 10c shows connecting arms 100 as described in FIG. 7a and FIG. 7b. However, any suitable connecting arms as described herein may be used.

Referring back to FIG. 1, the tubular member 12, 14 includes at least one external surface 70 which has been described hereinabove and an inner surface 208 which may form the cavity 16. At least one of the inner surface 208 and the external surface 70 may be coated with at least one layer of coating. The at least one layer of coating may include one substance or a combination of substances. The at least one layer of coating may include a glue, an adhesive, sealant or any suitable adhering substance. The glue may promote adhering the ends to be joined of the inserted biological structures and/or non-biological structures. The glue may facilitate temporary sticking together of the structures to be joined until the structures heal and join. In some non-limiting examples, the glue may be applied about the inner surface 208 of the proximal opening 20 of the tubular member 12, 14 in order to facilitate bonding of the cut ends of the structures to be joined. Non-limiting examples of suitable glues for use in the present invention include surgical glues, fibrin sealants, collagen based compounds, glutaraldehyde glues and hydrogels. The at least one layer of coating may include an antibiotic or any other suitable antibacterial substance. The at least one layer of coating may include at least one substance for promoting healing, a therapeutic agent, an anticlotting substance, a clotting substance, a vitamin, an antioxidant, an anti-inflammatory agent, an anesthetic agents, an anti-coagulant, an anti-restenosis agent, a thrombosis agent, an immunosuppressant agents, a dye, a movement retardation composition or combination thereof. The at least one layer of coating may be covered with at least one release liner. The release liner may prevent the coating from sticking before application and use of the device. The at least one release liner may be removed prior to use. The device may include a plurality of release liners, such as, but not limited to two release liners, a release liner for each part or tubular member, or section of the device. The inner surface 208 and/or the outer surface 70 may be coated with a different at least one layer of coating. In some non-limiting examples, the inner surface 208 may include at least one layer of coating and the outer surface 70 may not include at least one layer of coating.

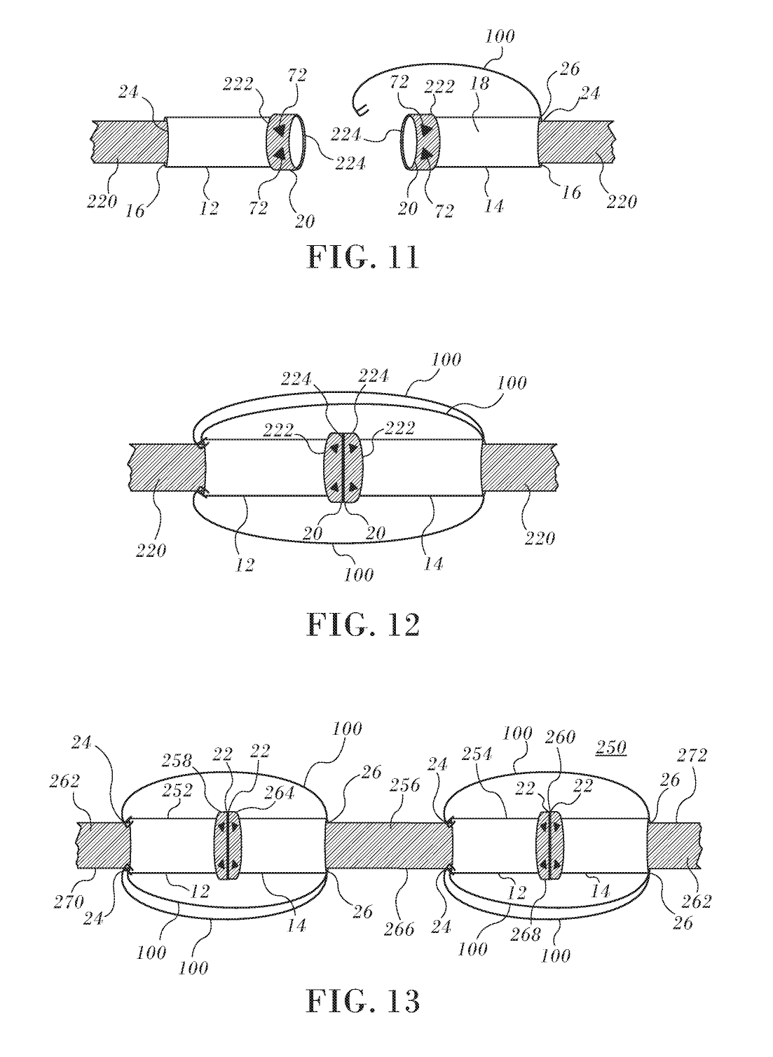

FIG. 11 shows a schematic view of two exemplary unconnected tubular members 12, 14 of a device 10 with at least one inserted biological structure according to an aspect of the present invention. As can be seen each of the two tubular members 12, 14 accommodates and holds a structure 220 within the cavity of the tubular member 12, 14, such as a biological structure 220. The tubular member 12, 14 is sized to accommodate within it only a portion of the biological structure 220, the portion which may be adjacent to the cut end to be joined. The end 222 of the biological structure 220 is everted over the edge of the proximal opening 20 and fixed with the protrusions 72 on the exterior surface of the tubular member 12, 14 facilitating an open end 224 of the biological structure 220 positioned at the proximal opening 20 of the tubular member 12, 14. Part of the biological structure 220, which extends from the end 224 at the proximal opening 20 until the distal opening 24 of the tubular member 12, 24 is accommodated inside the cavity 16 of the tubular member 12, 14 and the continuation of the biological structure 220 therefrom is not contained within the tubular member 12, 14. One of the tubular members 14 has at least one attached connection arm 100, which extends from the distal end 26 of the tubular member 14, above the body 18 of the tubular member 14 and in a direction towards the proximal opening 20 of the tubular member 14. The connecting arm 100 overhangs the tubular member 14 past the proximal opening 20. The connecting arm 100 is configured for connecting the two tubular members 12, 14 end 20 to end 20 to form a linear unitary device. FIG. 11 shows the at least one connecting arms 100 as arcuate arms with hooks, however this is not meant to be limiting and the at least one connecting arm 100 may be as described in FIGS. 7-8 or any other suitable connecting arm as described herein.

FIG. 12 shows a schematic view of two ends 224 of a biological structure connected with an exemplary connector device according to an aspect of the present invention. The device may be a device as described herein. As can be seen from FIG. 12 one end 222 of part of the biological structure 220 to be joined is inserted in a first tubular member 12 of the device. The end 222 to be joined is everted over the proximal opening 20 of the first tubular member 12. Inserted in the second tubular member 14 of the device is a second end 222 of part of the biological structure 220 to be joined. The end 222 of the second part of the biological structure 220 is everted over the proximal opening 20 of the second corresponding tubular member 14. The resulting two ends 224 fixed at the proximal openings 20 are in contact and a plurality of arched connector arm components 100 of the second tubular member 14 are hooked onto the first tubular member 12 for holding the two ends 224 together to join the two parts of the biological structure 220 and facilitating a unitary type device 140, such as described in FIG. 4d. FIG. 12 shows the at least one connecting arms 100 as arcuate arms with hooks, however this is not meant to be limiting and the at least one connecting arm 100 may be as described in FIGS. 7-8 or any other suitable connecting arm as described herein.

FIG. 13 shows a schematic view of an exemplary system 250 of the present invention to connect a plurality of structures. An exemplary system 250 may include a plurality of devices 252, 254. Each device 252, 254 may include a pair of corresponding tubular members 12, 14. The plurality of structures to be joined may include at least four ends to be joined. The system may connect a structure 256 to a first end 258 and a second end 260 of a cut biological structure 262. One non-limiting example shown in FIG. 13 is of a biological structure 262, which is a blood vessel 262 and a structure 256 which is a synthetic blood vessel graft 256. The figure shows a first device 252, The first device 252 may include a pair of corresponding tubular members 12, 14, including a first tubular member 12 and a second tubular member 14. The first device 252 may be configured for accommodating and joining the first end 258 of the biological structure 262, such as the cut blood vessel 262 to the first end 264 of the graft 256. The first tubular member 12 may hold the first end 258 of the cut blood vessel 262 and the second tubular member 14 may hold the first end 264 of the blood vessel graft 256. The first end 258 of the cut blood vessel 262 may be everted over the proximal end 22 of the first tubular member 12 and the first end 264 of the blood vessel graft 256 may be everted over the proximal end 22 of the second tubular member 14. A plurality of connecting arms 100, which are part of and disposed about the distal end 26 of the second tubular member 14 are attached to the distal opening 24 of the first tubular member 12 to form a connected first unitary device 252 with an end to end join of the first end 258 of the biological blood vessel 262 to the first end 264 of the blood vessel graft 256, which are positioned about the proximal ends 22 of the first tubular member 12 and the second tubular member 14 respectively. FIG. 13 shows the first end 264 of the graft 256 positioned at the proximal end 22 of the second tubular member 14 of the first device 252 and an extension 266 of the graft 256 extending from the distal end 26 of the second tubular member 14 of the first device 252. A second end 268 of the graft 256 may be accommodated in a second device 254. The second device 254 may be a device of the present invention as described herein and shown in FIGS. 1-8, 10-12. The second device 254 may be the same as the first device 252. The second device 254 may include a pair of corresponding tubular members 12, 14 featuring a first tubular member 12 and a second tubular member 14. To avoid repetition the parts of the second device 254 may be as described hereinabove for the first device 252. The second end 268 of the graft 256 may be accommodated in the first tubular member 12 of the second device 254, such that the second end 268 of the graft 256 is evened about the proximal end 22 of the first tubular member 12 of the second device 254. The second tubular member 14 of the second device 254 may accommodate the second end 260 of the cut blood vessel 262, such that the second, end 260 of the cut blood vessel 262 is everted about the proximal end 22 of the second tubular member 14 of the second device 254. The first tubular member 12 of the second device 254 with the everted second end 268 of the blood vessel graft 256 and the second tubular member 14 of the second device 254 with the evened second end 260 of the biological blood vessel 262 are contacted such that the second end 260 of the blood vessel 262 and the second end 268 of the graft 256 are in contact end to end. A plurality of connecting arms 100 disposed at the distal end 26 of the second tubular member 14 of the second device are attached to the distal opening 24 of the first tubular member 12 of the second device to form a second unitary device 254 with an end to end join of the second end 268 of the blood vessel graft 256 to the second end 260 of the biological blood vessel 262. FIG. 13 shows the at least one connecting arms 100 as arcuate arms with hooks, however this is not meant to be limiting and the at least one connecting arms 100 may be as described in FIGS. 7-8 or any other suitable connecting arm as described herein. The system 250 facilitates a new biological structure, which may include a first part 270 of a blood vessel joined to a first end 264 of a blood vessel graft 256, the length 266 of the blood vessel graft 256 and a second end 268 of the blood vessel graft 256 joined to the second part 272 of the blood vessel 262. The system 250 can be used to join any suitable structures and any suitable number of structures. In an example where more structures are joined, additional devices of the present invention may be employed.

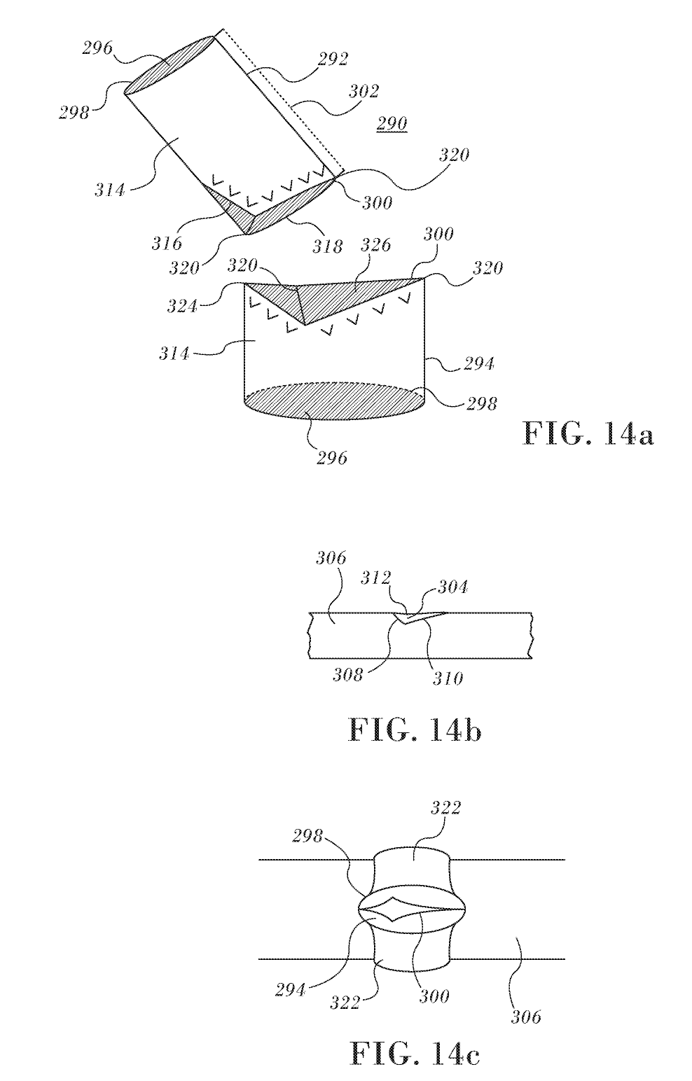

In one aspect the device of the present invention may be used in a side to end connection, such as in a bypass procedure for promoting optimal blood flows in a blocked blood vessel, such as a blocked artery. FIG. 14a shows a schematic view of an exemplary device 290 for use in a bypass procedure. The device 290 may include a pair of corresponding tubular members 292, 294, featuring a first tubular member 292 and a second tubular member 294. The first tubular member 292 may be configured for use with a graft and for accommodating a graft in the cavity 296 of the first tubular member 292. The first tubular member 292 may include a distal opening 298 and a proximal opening 300. The proximal opening 300 is the opening at which an end of the graft to be joined may be positioned. The first tubular member 292 may be the same or include at least some same features as described for the tubular member 12, 14 in FIG. 1. A difference from the bypass first tubular member 292 and the tubular member 12, 14 of FIG. 1 may be the proximal opening 300 of the bypass first tubular member 292. The proximal opening 300 may be a non-unit opening. At least a part of the proximal opening 300 may not be parallel to the distal opening 298. The proximal opening 300 may be an opening, which is angled and wherein at least parts of the opening are not perpendicular to the longitudinal length 302 of the tubular member 292. The proximal opening 300 may be configured for joining the graft to an artery, so that blood can flow from the artery and through the graft. The proximal opening 300 may be configured to provide blood flow in more than one direction, such as for example blood flow from the artery through the graft and vice versa and blood flow from the graft and across the restricted area of the artery. The bypass first tubular member 292 may be configured with a similar corresponding proximal opening 300 to the opening 304 in the artery 306.

The cut made in the artery 306 may result in an opening 304 in the artery 306, which may be non-uniform and which may have any suitable shape, and dimensions. In one non-limiting example. the surgeon may cut the artery to facilitate a kite like shaped opening of two triangles 308, 310 sharing the same base 312 as shown schematically in FIG. 14b. In such a non-limiting example the proximal opening 300 of the first tubular member may correspond to the shape of the opening 304 in the artery 306 and may be a folded kite shaped opening, wherein one triangular like part 316 is at an angle to the second triangular like part 318. One of the triangular parts 316 of the opening 300 may be formed from the body 314 of the tubular member 292 and the second part 318 of the opening 300 which may be triangular in shape may be formed at the proximal extremity 320 of the tubular member 292. At least one part 316 of the proximal opening 300 of the bypass first tubular member 292 may correspond to the first triangular opening 308 in the artery 306 or other shaped first opening and at least a second part 318 of the proximal opening 300 of the bypass tubular member 292 may correspond to a second triangular opening 310 or other shaped second opening in the artery 306.

The bypass first tubular member 292 may be configured with a proximal opening 300 corresponding to the opening 304 in the bypass second tubular member 294. The shape and angle of the proximal opening 300 of the bypass lust tubular member 292 may be configured to provide the correct shape and angle of the graft for optimal connection to the bypass second tubular member and the opening in the artery 306. The shape and angle of the proximal opening may facilitate any type of cut of the graft, which can be adapted by eversion over the proximal opening 300 to the correct shape and angle for optimal connection to the opening in the artery and without the need for cutting the graft accurately according to the opening in the artery 306.

A further difference between the bypass first tubular member 292 and the tubular member 12, 14 described in FIG. 1 may be the body of the tubular member. The body 18 of the tubular member 12 14 shown in FIG. 1 is a uniform body with a proximal opening 20 and distal opening 24, which are parallel to each other. The bypass tubular member 292 may include a body 314, which is not a uniform cylinder and which may have cutouts.

The corresponding tubular member 294 of the bypass device 290 may be for applying about a cut or opening 304 in the artery 306. The corresponding tubular member 294 of the bypass device 290 may be referred to as the second tubular member 294 of the bypass device 290 to distinguish it from the first tubular member 292 of the bypass device 290. The terms ‘first’ and ‘second’ being chosen arbitrarily for distinguishing purposes. The second bypass tubular member 294 may be the same or share at least some of the same features as a tubular member 12, 14 described in FIG. 1. A difference between the bypass second tubular member 294 and the tubular member 12, 14 described in FIG. 1 may be the proximal opening 300 of the bypass second tubular member 294. The proximal opening 300 may be a non-uniform opening. At least a part of the proximal opening 300 may not be parallel to the distal opening 298. The proximal opening 300 of the bypass second tubular member 294 may be configured according to the cut opening made in the artery. In some embodiments, the proximal opening 300 of the bypass second tubular member 294 is not configured according to the cut made in the artery, but provides any suitable cut opening in the artery with the shape and angle of the proximal opening 300 of the bypass second tubular member 294. As described above, in one non-limiting example the opening made in the artery may be a kite like shape of cite triangles sharing the same base. In a further non-limiting example, the cut/opening made in the artery may be in the shape of a cross, which may provide sufficient tissue to be everted over the proximal opening 300 and result in the opening in the artery adopting the same shape as the proximal opening 300 of the bypass second tubular member 294. The bypass second tubular member 294 may be configured to be attached onto and about the opening 304 in the artery 306. The second bypass tubular member 294 does not accommodate the artery in the cavity 296 of the second bypass tubular member 294, because the artery is not completely dissected and therefore cannot be inserted in a tubular member.

The second bypass tubular member 294 may sit on the artery 306. In some embodiments, the second bypass tubular member 294 may be configured with means 322 to facilitate sitting on the artery about the opening in the artery. Suitable means 322 may include, but is not limited to wing extensions 322 which may be shaped in line with the contour of the artery 306 as shown schematically in FIG. 14c. The wing extensions 322 may be disposed about the distal opening 298 of the second bypass tubular member 294. In some embodiments, the distal opening 298 may be a circular opening with wings 322 surrounding or partially surrounding the distal opening 298. In some embodiments, the distal opening 298 of the second bypass tubular member 294 may be shaped corresponding to the contour of the artery 306, with sides overhanging the artery 206 for anchoring the second bypass tabular member 294 to the artery. Such a distal opening 208 may differ from the distal opening of the tubular members 12, 14 described in FIG. 1. Referring back to the non-limiting example of FIG. 14a, the first part 324 of the proximal opening 300 in the second bypass tubular member 294 may be formed from a first part of the body 314 of the second bypass tubular member 294 and the second part 326 of the proximal opening 300 in the second bypass tubular member 294 may be formed from a second part of the body 314 of the second bypass tubular member 294. The first part of the body 314, may be cut out to form the corresponding first part 324 of the proximal opening 300. The second pan of the body 314, may be cut out to form the corresponding second part 326 of the proximal opening 300. In the non-limiting example shown in FIG. 14a, the two parts 324, 326 of the proximal opening 300 form a lute shape of two triangular cut outs of the body 314 extending from the proximal extremity 320 of the second tubular member 294 into the body 314. The two parts 324, 326 of the opening 300 of the second bypass tubular member 294 may both extend from the proximal extremity 320 of the second bypass tubular member 294 and may include an edge surrounding the opening. In one non-limiting example the diameter of the proximal opening 300 may be less than the diameter of the tubular member 294 and the diameter of the distal opening 298 of the second bypass tubular member 294.

A further difference between the bypass second tubular member 294 and the tubular member 14 described in FIG. 1 may be the body of the tubular member. The body 18 of the tubular member 12, 14 shown in FIG. 1 is a uniform body with a proximal opening 20 and distal opening 24, which are parallel to each other. The bypass tubular member 294 may include a body 314, which is not a uniform cylinder and which may have cutouts.

The proximal opening of the first bypass tubular member corresponds to the proximal opening of the second tubular member. The proximal opening 300 of the first bypass tubular member and the proximal opening 300 of the second bypass tubular member may be shaped and configured for a predetermined angle of connection of a bypass structure to the opening in the artery. The proximal opening 300 of the first bypass tubular member 292 and the proximal opening 300 of the second bypass tubular member 294 may be shaped to provide an end to side connection.

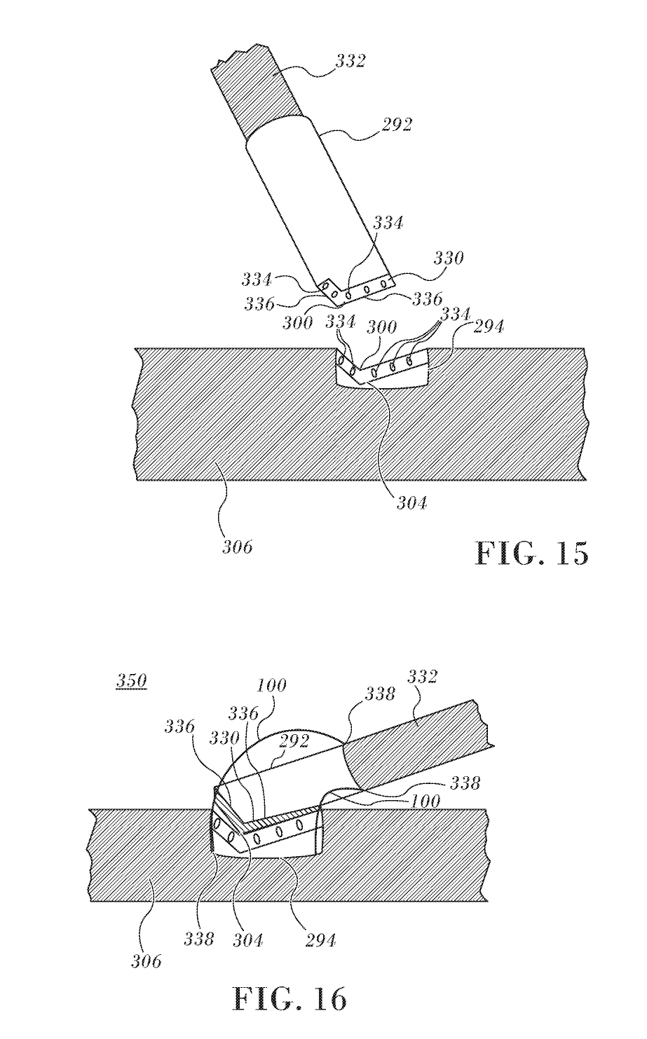

FIG. 15 shows schematically a first end of a graft attached to a first bypass tubular member and a Second bypass tubular member attached to an opening in an artery according to an aspect of the present invention. A first end 330 of a graft 332 may be inserted in the cavity of the first bypass tubular member 292, so that the first end 330 of the graft 332 is positioned at the proximal opening 300 of the first bypass tubular member 292. The first end 330 of the graft 332 may be everted over the edge of the proximal opening 300 of the first bypass tubular member 292. The first bypass tubular member 292 may include protrusions 334 on the external surface of the first bypass tubular member 292 for fixing the first end 330 of the graft 332 to the first bypass tubular member 292 and for facilitating a substantially even edge 336 of the opening of the graft 332 for connection to the artery. The protrusions 334 may be as described hereinabove. The protrusions 334 may be spaced equidistance from the different sides of the proximal opening 300. The shape of the proximal opening 300 of the first bypass tubular member 292 may configure the correct angle, shape and size of the first end 330 of the graft 332 and/or exposed first end opening of the graft 332 for optimal connection to the opening in the artery. Advantages of the present invention include a surgeon or other suitable medical practitioner not needing to cut the graft according to the opening in the artery and not needing to calculate and cut according to the angle needed for optimal connection.

The corresponding bypass tubular member 294 referred here as the second bypass tubular member 294 may be inserted on top of the opening 304 in the artery 306 and the edges of the cut opening 304 may be everted over the proximal opening 300 of the second bypass tubular member 294. The edges of the opening 304 may be fixed to the second tubular member 294 with protrusions 334 on the external surface of the second bypass tubular member 294 facilitating a substantially even edge of the opening 304 for connection to the graft 332. The protrusions 334 may be as described hereinabove. The protrusions 334 may be spaced equidistance from the different sides of the opening 300. The second bypass tubular member 294 may provide the correct angle/s for the opening 304 in the artery 306 for coned positioning and connection to the graft 332. A surgeon does not need to ensure the angle or shape of the cut as the second bypass tubular member 294 confers the correct angles, shape and sin of the opening 304 in the artery 306 for optimal bypass connection. The second bypass tubular member 294 may provide an anchor on to which the first bypass tubular member 292 can be connected.

FIG. 16 shows schematically a first bypass tubular member 292 attached to one end 330 of a graft 332 and a second bypass tubular member 294 attached to an opening 304 in an artery 306, wherein the first bypass tubular member 292 and the second bypass tubular member 294 are connected together according to an aspect of the present invention. The first bypass tubular member 292 may connect to the second bypass tubular member 294 at an angle, for contact of the opening/s 304 of the artery with the exposed opening/s 336 of the graft to facilitate an open unrestricted pathway for blood flow between the artery and graft. Connection may facilitate connection of the walls of the opening 304 of the artery 306 with the corresponding walls of the exposed opening 336 of the graft 332. Connection arms 100 as described herein may be a fixed component of the first bypass tubular member 292 or the second bypass tubular member 294 and may be fixed at the distal end 338 of the tubular member. In some embodiments, the connection arms 100 may be fixed to the bypass tubular member 292 in which, the graft is inserted. Although FIG. 16 shows the connection arms 100 as arched, the connection arms 100 may be linear and may be any suitable connection arms, such as those described in FIGS. 7-8. The connection arms 100 may be configured to attach to the corresponding other tubular member part of the bypass tubular device, which does not include the connection arms 100. The attachment of the at least one of a plurality of connection arms 100 to the corresponding other tubular member may facilitate a unitary bypass device 350, which includes the first bypass tubular member 292, the second bypass tubular member 294 and the protruding edge of the artery and the protruding end of the graft therebetween. The unitary bypass device 350 may not be a linear device, but the first tubular member 292 and the second tubular member 294 may be at an angle to each other. The connection of the first bypass tubular member 292 and, the second bypass tubular member 294 may facilitate blood flow from and to the artery 306 via the graft 332.

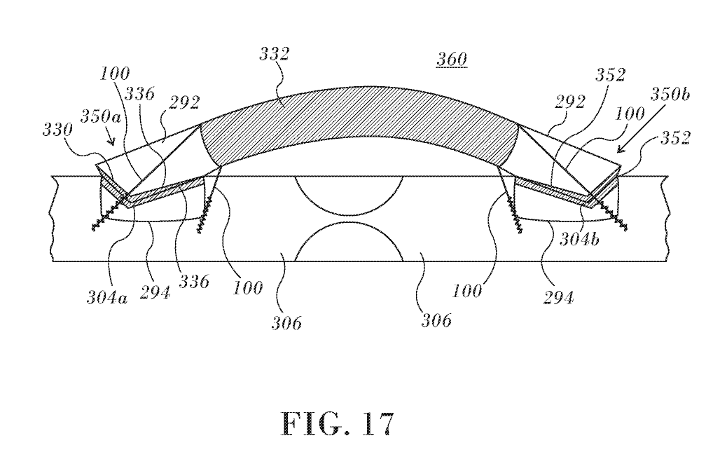

In a bypass procedure a first end of a graft and a second end of a graft may be attached to two openings in an artery. One opening in an artery may be before a blockage in the artery and a second opening in the artery may be after a blockage in the artery. FIG. 17 shows schematically an exemplary system 360 of the present invention used in a bypass procedure according to an aspect of the present invention. A system 360 of the present invention for use in a bypass procedure, may include a plurality of devices 350a, 350b of the present invention as described hereinabove and shown in FIGS. 14-16. A first device 350a may be for connecting a first opening 304a in an artery 306 to a first exposed end 336 of a first end 330 off graft 332 and a second device 350b may be for attaching a second end 352 of a graft 332 to a second opening 304b in an artery to provide blood flow through the artery 306 via the graft 332. Each of the two devices 350a, 350b may include a first bypass tubular member 292 and a second bypass tubular member 294, with connection arms 100 and corresponding connection means as described herein. The proximal openings 300 of the first bypass tubular member 292 and the second bypass tubular member 294 of the first device 150a and the second device 350b may be configured as described hereinabove and shown in FIGS. 14-16. The tubular members 292, 294 of the devices 350a, 350b may standardize, the connection of the artery 306 to the graft 332 by adapting the openings 304a, 304b in the artery 306 and the openings 336, 352 in the graft 332 to the proximal openings 300 of the devices 350a, 350b.

The device and the device components may be made from an suitable material. The material may be biocompatible and non-toxic. The material may be a material which has sufficient properties of flexibility to impart flexibility on the device and the device components. The device and the device components may have a flexibility such that they can expand and contract according to the changing dimensions of an inserted structure. Non limiting examples of materials which may be used include nitinol, alloys of titanium and nickel, stainless steel, platinum, gold, silver, copper, zinc, ceramic, polytetrafluoroethylene (PTFE), polyethylene, urethane, silicone, nylon, polyester, polypropylene, fabric, gut, tissue graft and, combinations thereof. In some embodiments, the device and parts thereof are made of a shape-memory material, such as, but not limited to nitinol. The device and parts thereof may be constructed from the same material or from different materials. The material may be disposable. The material may be absorbent or non-absorbent. The material may be porous and may have different porosities. The device and/or parts thereof ma be reusable. In some embodiments, the device is configured for one time use.

The device may be used for application to blood vessels, lymphatic vessels, ligaments, or any other suitable biological structure or synthetic structure of any suitable size. The same connector device may be used to connect structures such as blood vessels of different sizes. This may be achieved, as the internal dimensions of the device may be adjusted as described hereinabove in order to accommodate structures of different dimensions. The connector devices of the present invention may be designed and constructed in a range of dimensions, to be suitable for different biological structures and different ranges of size of biological structures.

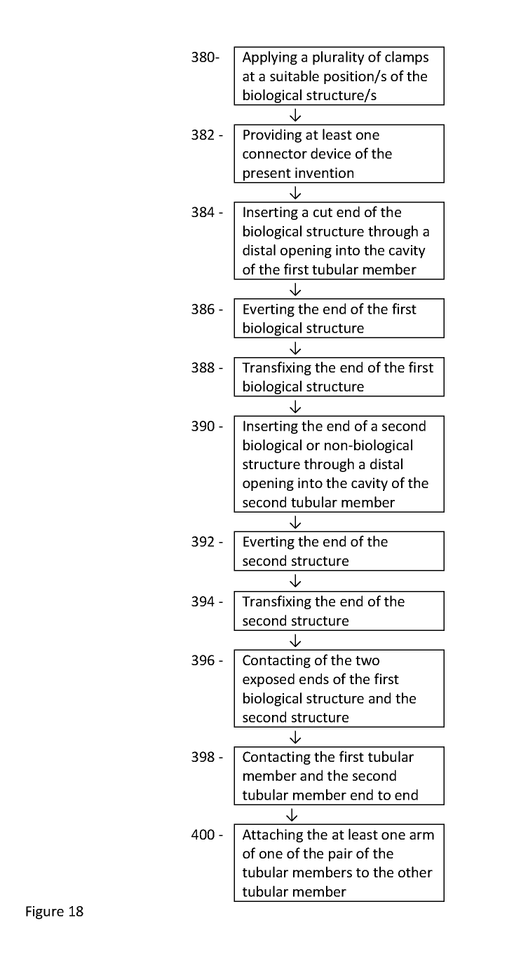

The present invention provides a method of connecting a biological structure and at least one other structure. FIG. 18 shows a flow chart of an exemplary method of using the device of the present invention to connect a biological structure and at least one other structure according to an aspect of the present invention. A plurality of clamps or equivalent blood restriction elements may be applied at a suitable position/s of the severed biological structure to temporarily stop or reduce blood flow and bleeding from the severed ends 380. At least one device of the present invention may be provided 382. The device may be chosen according to its size in relation to the structures to be joined. The device may include a first tubular member and a corresponding second tubular member. The first tubular member may include a first opening at a proximal extremity of the tubular member, a second opening at a distal extremity of the tubular member, a central cavity within the tubular member for accommodating the structure and a plurality of spaced apart protrusions for holding an end of the structure everted over the proximal extremity of the tubular member. The second tubular member may include a first opening at a proximal extremity of the tubular member, a second opening at a distal extremity of the tubular member, a central cavity within the tubular member for accommodating the structure, a plurality of spaced apart protrusions for holding an end of the structure everted over the proximal extremity of the tubular member and at least one arm for connecting the first tubular member to the second tubular member.

The method may include a user inserting into the cavity of the first tubular member, through the distal opening, the first biological structure or a part thereof 384. The method may including evening the end of the first biological structure or part thereof over the opening at the proximal extremity of the first tubular member 386. This may be done in order to prevent the biological structure from moving away from the opening of the device, such as due to spasms or contraction. In addition. eversion provides an optimal part of the biological structure positioned at the proximal opening to be joined. A severed end may not be even and there may be damage at the edges of the extremity, which may be prone to tear. Eversion may provide a more even and undamaged part of the biological structure to join, which may result in a better joining of the two structures. The method may include fixing the everted end of the first biological structure or part thereof to the tubular member 388. Fixing may include transfixing the tissue with the plurality of spaced apart protrusions, such as spikes. The spikes may pierce the tissue or synthetic equivalent to hold the structure in place. The protrusions may be positioned on the external surface of the extended body of the first tubular member at or near to the proximal opening.

The method may include inserting into the cavity of the second tubular member, through the distal opening, a second structure to be joined to the first biological structure 390. The second structure may be part of the same biological structure inserted into the first tubular member. The second structure may be a different structure, which may be a biological structure or which may be a non-biological structure or an external biological structure. The term ‘non-biological structure’ as used herein may include any structure which is not found naturally in a human or animal body and/or whose source is not biological. Non-limiting examples of non-biological structures may include synthetic grafts, catheters, tubes, wires, inlets of machinery, outlets of machinery and combinations thereof. The term ‘external biological structure’ as used herein may include an structure, which may be biological taken from an external source horn the host. Non-limiting examples of external biological structures may include biological structures or parts thereof from a different host or grown in vitro or grafts for transplanting. The end of the second structure may be everted over the proximal opening of the second tubular member 392. In one non-limiting example the first biological structure may be one part of a cut blood vessel and the second structure may be a second part of the cut blood vessel.

The method may include fixing the everted end of the second structure or part thereof to the tubular member 394. Fixing may include transfixing the tissue or synthetic equivalent with the plurality of spaced apart protrusions, such as spikes. The spikes may pierce and/or spear the tissue or synthetic equivalent to hold the structure in place. The protrusions may be positioned on the external surface of the extended body of the second tubular member at or near to the proximal opening.

A user may contact the opening of the everted end of the first biological structure positioned at the proximal opening of the first tubular member with the opening of the everted end of the second structure positioned at the proximal opening of the second tubular member 396. The contact may be end to end to facilitate a continuous internal pathway of the joined structures. The user may contact the two tubular members end to end 398. The user may connect together the two tubular members of the device to securely fix together the two ends of the inserted structures for optimal joining and healing. The user may manipulate the at least one arm of the second tubular member to hook and attach to a corresponding means on the distal extremity of the first tubular member to connect the first tubular member to the second tubular member and to connect the end of the first biological structure to the end of the second structure 400. In an embodiment wherein the means to connect the first tubular member to the second tubular member of a pair of tubular members includes use of an arched at least one arm with a hook as described herein manipulating may include pushing and pulling the at least one arch to extend and reduce the diameter of the arm. In order to hook the distal extremity of the first tubular member, a user may have to push the two tubular members together and pull the extension of the arm so that it is able to reach the distal opening of the fist tubular member. Manipulating may include inserting a prong of the at least one arm through the distal opening of the first tubular member and into the cavity of the first tubular member. The tension of the arch of the arm may provide the hooking force to keep the arm attached to the first tubular member. In an embodiment wherein the means to connect the first tubular member to the second tubular member of a pair of tubular members includes the at least one arm as described in FIGS. 7a-7b manipulating may include pushing downwards the at least one arm, which is fixed on one tubular member until it is in line with a raised slit on the corresponding tubular member, the at least one slit at a lower elevation than the toothed arm, to facilitate inserting the at least one arm through the at least one slit. Manipulating may include inserting the free end of the at least one arm and unidirectional teeth disposed thereon, into and through the corresponding at least one slit on the corresponding tubular member. Manipulating may include repeating with each of the at least one arms. Manipulating may include contacting the two parts of the device, with a simultaneous additional length of the arm proceeding through the slit or manipulating may include pulling the length of the arm through the slit until the two proximal openings of the device are in contact or manipulating may include a combination thereof. In an embodiment wherein the means to connect the first tubular member to the second tubular member of a pair of tubular members includes the at least one arm as described in FIGS. 8a-8b, manipulating may include contacting the proximal end of one of the tubular members with the proximal end of the corresponding tubular member. Manipulating, may include pushing the at least one arm fixed to one of the tubular members upwards, until the at least one arm is in line with at least one protrusion on the corresponding tubular member. Manipulating may include inserting the free end of the at least, one protrusion into an opening of a plurality of spaced apart openings disposed along the length of the at least one arm. A user may repeat the manipulating and connecting for each at least one arm of a plurality of arms. In an embodiment, wherein the device includes four connecting arms, a user may connect four arms to the first tubular member. In the description of FIG. 18, the second tubular member is described with a connecting arm and the first tubular member is described without a connecting arm. However, FIG. 18 also applies to a device, wherein the first tubular member may include a connecting arm and the second tubular member may not include a connecting arm. FIG. 18 may also apply to a device wherein both tubular members of a corresponding pair do not include a connecting arm, in which case a standalone connecting arm may be used and attached to one of the two tubular members and then used as described herein.

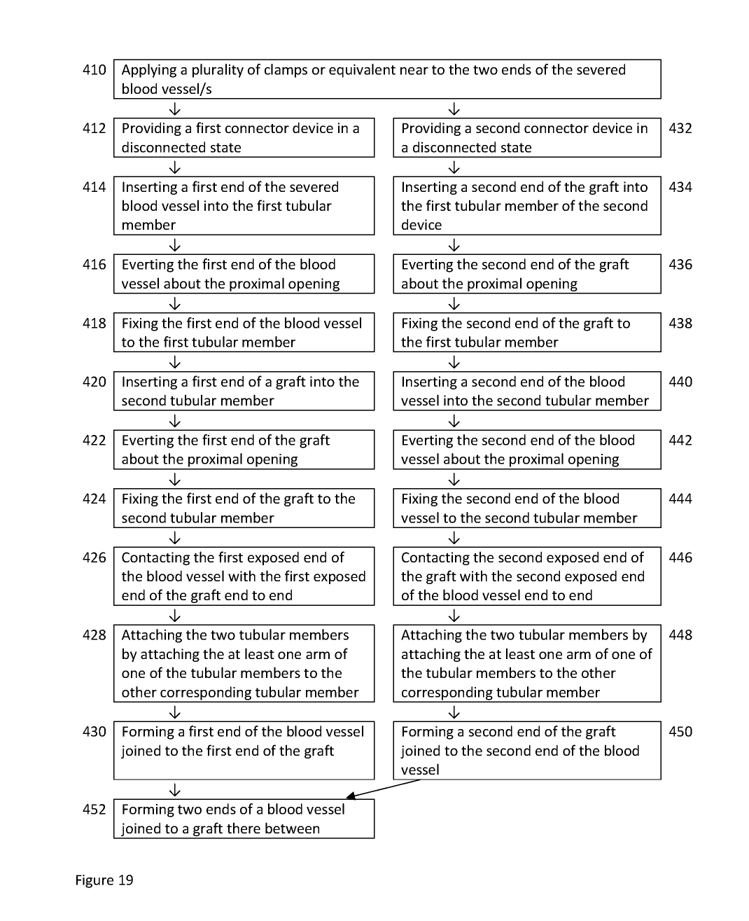

The present invention provides a method of connecting a first end and a second end of a graft to a first end and a second end of a cut blood vessel. FIG. 19 shows a flow chart of an exemplary method of connecting a graft to a cut blood vessel using a plurality of devices of the present invention according to an aspect of the present invention. A plurality of clamps or equivalent blood restriction elements may be applied, at a suitable position/s of both parts of the severed blood vessel to temporarily stop or reduce blood flow and bleeding from the severed ends 410.

The method may include providing a first device of the present invention 412. The connector device may be provided in a disconnected state or may be disconnected into a separate pair of tubular members. The device may be a device as described hereinabove in FIGS. 1-13 and to avoid repetition will not be repeated here. The device may be chosen according to its size in relation to the blood vessel to be joined. The first device may include a first tubular member and a second tubular member. A user may insert the first end of the cut blood vessel through the distal opening of the first tubular member of the first device into the cavity until the proximal opening of the first tubular member of the first device 414. The end of the cut blood vessel may overextend the proximal opening to facilitate a user everting the first end of the cut blood vessel over the proximal opening of the first tubular member of the first device 416. A user may fix the everted first end of the cut blood vessel using the plurality of spaced apart protrusions, such as spikes 418. The user may spear the everted tissue with the spikes to transfix the first end of the cut blood vessel, such that an open end of the cut blood vessel is positioned at the proximal opening of the first tubular member of the first device. In an alternative embodiment, a user may fix and hold the end of the structures to be joined using a different method than eversion. A user may insert the first end of the graft through the distal opening of the second tubular member of the first device into the cavity until the proximal opening of the second tubular member of the first device 420. A user may evert the first end of the graft over the proximal opening of the second tubular member of the first device 422. A user may fix the everted first end of the graft with the plurality of spaced apart protrusions, such as spikes 424. In an alternative embodiment, a user may fix and hold the end of the structures to be joined using different method than eversion. A user may contact the everted end of the first end of the cut blood vessel with the everted first end of the graft end to end 426. A user may manipulate each of the at least one arms of the tubular member on which the at Last one arms are fixed, to attach to the other corresponding tubular member of the first device to connect the first tubular member of the first device to the second tubular member of the first device and to connect the first end of the blood vessel with the first end of the graft 428. Manipulation may depend on the attachment means on the at least one arm and may be as described hereinabove for FIG. 18 and which will not be described here to avoid repetition. The attachment of the pair of tubular members of the first device may facilitate, forming a first end at the blood vessel joined to the first end of the graft 430. The method may include providing a second device 432. A device, wherein the tubular members are connected may be disconnected before use. The device may be provided in a disconnected state. The second device may be a device as described hereinabove in FIGS. 1-13 and to avoid repetition will not be repeated here. The second device may include a first tubular member and a second tubular member. A user may insert the second end of the graft through the distal opening of the first tubular member of the second device and into the cavity until the proximal opening of the first tubular member of the second device 434. The second end of the graft may overextend the proximal opening such that a user may evert the second end of the graft over the proximal opening of the first tubular member of the second device 436. A use may fix the everted second end of the graft using the plurality of spaced apart protrusions, such, as spikes 438. The user may spear the everted tissue with the spikes to transfix the second end of the graft, such that an open end of the graft is positioned at the proximal opening of the first tubular member of the second device. A user may insert the second end of the cut blood vessel through the distal opening of the second tubular member of the second device and into the cavity until the proximal opening of the second tubular member of the second device 440. A user may evert the second end of the cut blood vessel over the proximal opening of the second tubular member of the second device 442. A user may fix the everted second end of the cut blood vessel with the plurality of spaced apart protrusions, such as spikes 444. In an alternative embodiment, a user may fix and hold the end of the structures to be joined using a different method than eversion. A user may contact the everted end of the second end of the graft with the everted second end of the cut blood vessel, end to end 446. A user may manipulate each of the at least one arms of the tubular member of the second device on which the at least one arms are fixed, to attach to the other corresponding tubular member of the second device in order to connect the first tubular member of the second device to the second tubular member of the second device and to connect the second end of the graft with the second end of the cut blood vessel 448. Manipulation may depend on the attachment means on the at least one arm and may be as described hereinabove for FIG. 18 and which will not be described here to avoid repetition. Attaching the two parts of the second device may facilitate forming a second end of the graft joined to the second end of the blood vessel 450. The attaching of the two parts of the first device and the two parts of the second device may facilitate forming two ends of a blood vessel joined to a graft positioned there between 452. The order of the steps of the method is not meant to be limiting and may be in any suitable order. In some examples, a plurality of the steps of the method may be done simultaneously. The description of FIG. 19 may include an example of the second tubular member including the at least one connecting arm and the first tubular member without a connecting arm. FIG. 19 may also include an example wherein the first tubular member includes the at least, one connecting arm and the second tubular member does not include a connecting arm. FIG. 19 may also apply to a device wherein both tubular members of a corresponding pair do not include a connecting arm, in which case at least one standalone connecting arm may be used and attached to one of the two tubular members and then used as described herein.

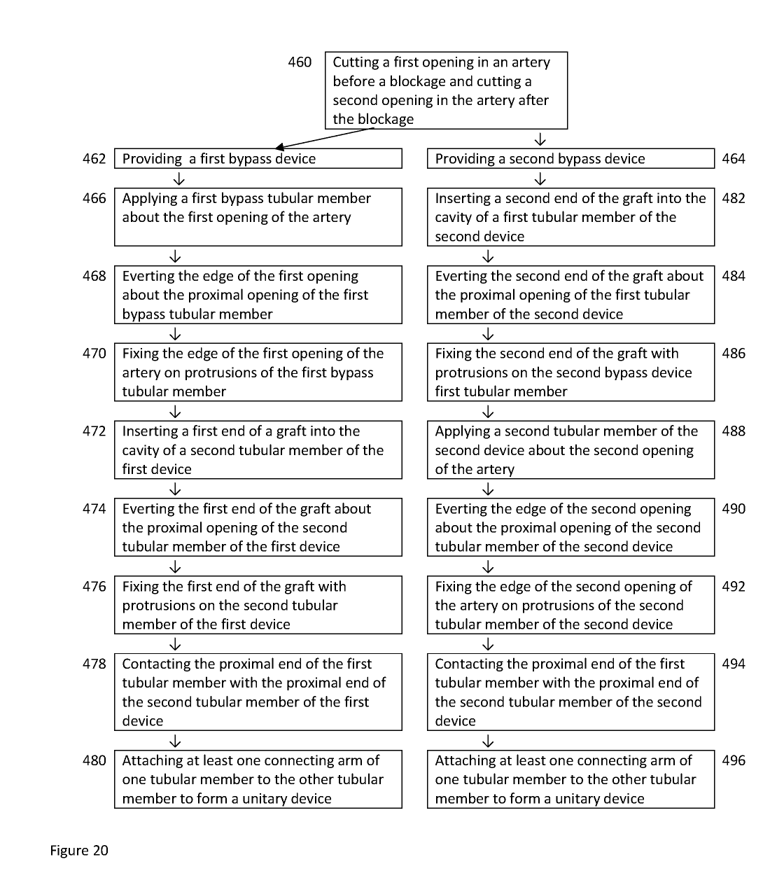

The device and system of the present invention may be used in a bypass procedure. FIG. 20 shows a flow chart of an exemplary method of using a system of the present invention in a bypass procedure according to an aspect of the present invention. The system may include a plurality of bypass devices as described herein. A user may cut a blocked blood vessel, such as for example an artery at a suitable position on a first side of a blockage before the blockage of an artery and a user may out a blocked blood vessel, such as an artery at a suitable position on a second side of the blockage after the blockage 460. The cut in the artery may result in an opening. The artery may not be fully dissected. The opening may have any suitable shape to provide sufficient tissue for eversion over the device. The method may include providing a graft, wherein the graft includes a first, end and a second end. The method may include providing a first bypass device of the present invention 462 and providing a second bypass device of the present invention 464. Each of the first bypass device and second bypass device may include a first bypass tubular member and a corresponding second bypass tubular member as described herein in FIGS. 14-16. A user may apply a first bypass tubular member of a first device about the opening on the first side of the artery 466. The edges of the opening in the artery may be everted over the proximal opening of the tubular member 468. The edges of the opening in the artery may be fixed to the first bypass tubular member by protrusions on the external body of the first bypass tubular member 470. The proximal opening of the first bypass tubular member may provide the size, shape and angle required for an optimal connection to the graft as described hereinabove. A user may apply a corresponding second tubular member of the first device about the first end of the graft, by for example inserting the graft through the cavity of the second tubular member until the proximal opening 472. A user may evert the first end of the graft about the proximal opening of the second tubular member of the first bypass device 474. A user may fix the first end of the graft to the second tubular member with protrusions on the external body of the second bypass tubular member 476. The shape of the proximal opening of the second bypass tubular member may provide the required size, shape and angle of the first end of the graft for optimal connection to the opening on the first side of the artery. A user may contact the opening of the artery fixed at the proximal end of the first bypass tubular member with the first end of the graft positioned and fixed at the proximal end of the second bypass tubular member, by for example contacting the proximal opening of the first bypass tubular member with the proximal opening of the second bypass tubular member 478. A user may attach at least one connecting arm, which is part of one of the bypass tubular members to the corresponding bypass tubular member 480. In one embodiment, the at least one connecting arm may be part of the tubular member attached to the graft and the end of the at least one connecting arm may attach to the distal end of the first bypass tubular member about the artery opening. The attaching of the at least one connecting arm may be according to the attachment means on the at least one arm as described herein for FIG. 18 and which is not described here to avoid repetition. A user may repeat the connecting with each of the at least one connecting arms. Connecting the first bypass tubular member to the second bypass tubular member may facilitate a unitary device, which provides blood flow through the artery via the graft. The first bypass tubular member and the second bypass tubular member may be configured as described herein to expand and contract according to the expansion and contraction of the opening on the first side of the artery and the expansion and contraction of the graft, which may be affected by the changes in blood flow.

A user may repeat steps 466-480 in steps 482-496 with a second bypass device to join the opening on the second side of the blocked artery cut after the blockage and the second end of the graft. This method facilitates bypassing of the blockage in the blocked artery by connection of the bypass blood vessel graft to the blocked artery with a plurality of multi-tubular devices of the present invention. The order of the steps of the method is not meant to be limiting and may be in any suitable order. It is envisioned that any techniques and procedures, preparations and parts of the protocol used in typical bypass procedures may be included in a method herein where suitable.

A user may treat the biological tissue prior to using the connector device of the present invention. In one aspect, the device may include at least one composition, which may treat the tissue as described herein.

The device or parts thereof of the present invention may remain connected to the biological tissues until the tissues join or rejoin or are healed. The device or parts thereof, may be removed after the tissues have joined. In some aspects, the device or parts thereof may remain in the body. Body tissue may grow and/or form over the device of the present invention. In some aspects, the device may degrade after the biological tissue/s have joined.

In one aspect, the connector device of the present invention is relatively facile for use by a user. A user may be a doctor, a surgeon, a nurse, a medical technician, a veterinarian or any suitable medical professional or individual. The device may be used by more than one user. In one non-limiting example one medical professional may insert a biological structure into one tubular member of a device, whilst a second medical professional inserts a second structure to be joined into a second tubular member of the multi-part device. In a non-limiting example, one medical professional may apply one device, whilst another medical professional may apply a second device. Alternatively, one medical professional may apply all parts of a device and/or all devices of the present invention in a procedure.

The user may employ his hands and/or any suitable equipment, tool or aid to use in combination with the device of the present invention. Non-limiting examples of suitable tools and aids include pincers, forceps, clamps, scissors, wires, tubes, computers, robots, magnifiers, drugs, antiseptic, cleaning agents, heat, cold, ice and sutures. A user may perform any procedure necessary prior to. during and after use of the device of the present invention. In one non-limiting example. wherein a blood vessel has been cut, a clamp or a plurality of clamps or any equivalent may be applied at a suitable position of both parts of the severed blood vessel to temporarily stop or reduce blood flow. A user may select a device of the present invention with dimensions suitable for optimally accommodating the structures to be inserted and joined.

The device of the present invention may be used to join a biological tissue to another biological tissue, or to join two parts of a biological tissue together or to join a biological tissue to a non-biological structure. The biological tissues may have been cut or damaged or may be in an unconnected state for any reason. The device may be used to join biological tissues that have been severed with any type of cut, such as but not limited to a cross-sectional cut, a straight cut, an angled cut and a combination thereof The biological tissue/s to be joined may be located internally in the body of a patient. In one aspect, the device of the present invention may be configured to be used externally to connect severed or injured biological structures found externally, such as but not limited to fingers and toes. The device may be employed in this way on for example a severed finger for joining the parts of the finger or as an initial pretreatment before connection of the finger using other means. The connector device may be packaged in sterile packaging, which may be opened before use. The device may be inserted manually or may be inserted via suitable insertion means which may be employed in for example keyhole surgery.

A user may apply more than one connector device of the present invention according to need. The more than one connector devices may be applied to different parts of the same biological tissue or to different biological tissues. Application of the device of the present invention may be one step or any suitable number of steps of any suitable surgical or medical procedure.

The device of the present invention may be used instead of other methods of connecting biological tissues or in addition to such methods. In one aspect, the device of the present invention may be used in addition to suturing and may augment suturing. Such a combination of methods is useful in a case where there is a need for reinforcement such as when there is a danger of stitches rupturing.

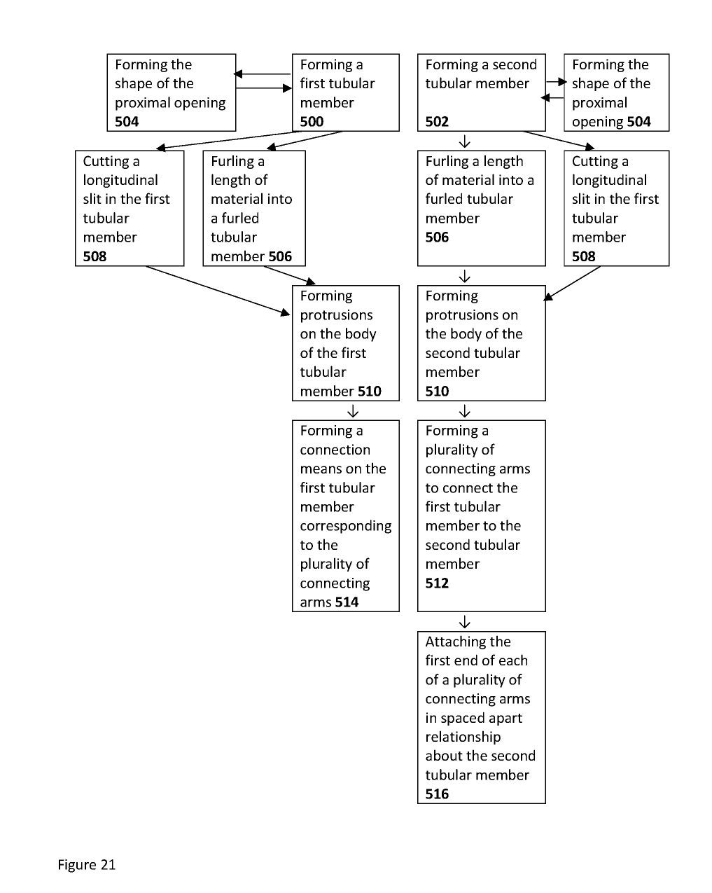

The present invention provider a method of production of a device for connecting at least one biological structure to at least one structure. FIG. 21 shows a flow chart of an exemplary method of constructing a device according to an aspect of the present invention. The method may include forming a first hollow tubular member from a suitable material 500. One non-limiting example of a suitable material may be nitinol. The hollow tubular member may include a first opening at a proximal extremity and a second opening at a distal extremity. The method may include forming a second hollow tubular member from a suitable material, such as but not hunted to nitinol 502. The second hollow tubular member may include a first opening at a proximal extremity and a second opening at a distal extremity.

In one embodiment, the method may include cutting the proximal opening of each of the first tubular member and the second tubular member to form an angled elliptical proximal opening 504. In an alternative embodiment, the proximal opening may not be cut and is uniform and perpendicular to the body of the tubular member.

In one embodiment the first tubular member and the second tubular members are furled tubular members. A furled tubular member may be made by curling a length of material to form a tubular member with an inner cavity surrounded by the length of material and with a diameter sized to accommodate a biological structure of a certain size 506. The flexibility of the material may be sufficient so that when the biological structure expands the tubular member unfurls to accommodate the larger diameter of the biological structure. The tubular member may be curled such that the degree of unfurling will still result in a tubular member sufficiently furled so that the inner cavity is surrounded by the length of material. The flexibility and properties of the material used to make the tubular member may be sufficient so that the tubular member refurls according to a contracting inserted structure.

In an alternative embodiment, the first tubular member and the second tubular member are not furled, but are tubular members which include a longitudinal slit. The tubular members may be cut longitudinally to afford a gap of less than about ten microns 508.

The method may include forming protrusions for transfixing an everted part of the end of an inserted structure to be joined 510. The protrusions may protrude out of the external surface of the tubular members. Forming protrusions may include punching a plurality of spikes adjacent the proximal extremity of the first hollow tubular member and the proximal extremity of the second hollow tubular member.

The method may include forming a plurality of connection arms on one of the tubular members 512 and forming a corresponding connection means on the other tubular member 514 of the pair of corresponding tubular members. The method may include attaching one end of a plurality of spaced apart arms in spaced apart relation about the distal extremity of one of the hollow tubular members facilitating a plurality of arms fixed at the distal extremity and extending from the distal extremity to beyond the first proximal extremity of the hollow tubular member 516. The attaching may be done using a technique, such as welding or gluing. In some embodiments, the at least one arm is integrally formed with the tubular member and is constructed from an extension of the tubular member.

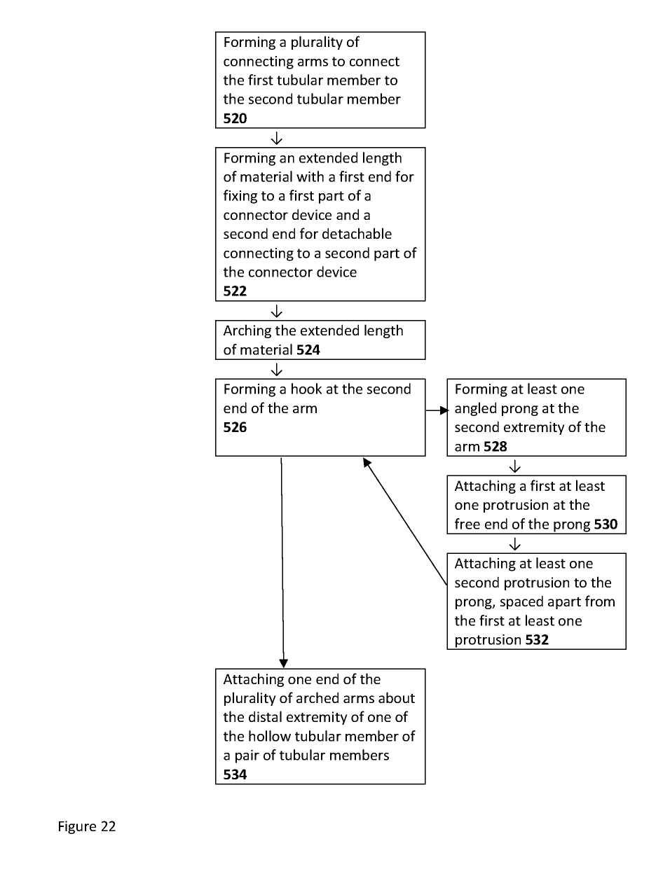

The present invention provides a method of production of connection means for connecting one tubular member to the other tubular member of a pair of tubular member. FIG. 22 shows a flow chart of an exemplary method of constructing the connection means according to an aspect of the present invention.

In one embodiment forming a plurality of connection means may include firming a plurality of connecting arms for connecting one tubular member to a corresponding second tubular member of a pair of tubular members 520. An arm may be made by forming an extended, strip of material, where the extended length includes a first end for attachment to a first tubular member of a connector device and a second end for detachable connecting to a second tubular member of the connector device 522. The material may be the same material as the tubular members are formed from, such as, but not limited to nitinol. The area may be arched, such that the arch may be manipulated to form a diameter equivalent to the combined length of the two tubular members of a pair of tubular members to be joined and the length of the protruding everted parts of the structure/s to be joined, which are protruding over the proximal extremity of each tubular member of the pair of tubular members 524. The arm may be made from a material with a limited flexibility, such that the end of the arm can be pulled, but the tension of the arm provides a spring type property for reverting to the original diameter. The spring type property may be used to thinly hook the arm to the second tubular member.

The forming a plurality of arched arms may include forming a hook at the second end of the arm, the second end defined as the end which is not attached before connection of a first tubular member to a second tubular member of a pair of tubular members 526. Forming the hook may include forming at least one prong at an extremity of the arm, wherein the at least one prong may be an extension of the arm, angled towards the arch and where the prong includes a length of prong and a free end 528 The forming may include forming and/or attaching a first at least one protrusion to the at least one prong 530. The fist at least one protrusion may be formed from a length of material, which is positioned at the free end of the prong to protrude at an angle from the prong towards the first end of the arm. The first at least one protrusion may be configured to hook the distal end of the second part of the deuce (the part which does not include an attached connector arm) and to attach into the cavity of the second part of the device to detachably fix the moveable arm to the second part of the device. Forming a plurality of at least one arms may include forming a second at least one protrusion from a length of material and positioning it spaced apart from the first at least one protrusion along the length of the prong so that it protrudes at an angle towards the first end of the arm 532. The second at least one protrusion may be made longer than the first at least one protrusion, such that it is adapted to allow the first at least one protrusion to attach into the cavity of the second part of the device (the part which does not include an attached connector arm) and to prevent displacement of an additional part of the arm from entering the cavity of the second part of the device.

In some embodiments the method may include forming at least one downward protrusions for preventing displacement. The at least one downward protrusion may be formed as a leg from a suitable material, such as from the same material the at least one arm is made from, which in one non-limiting example may be nitinol. The at least one downward protrusion may be sized so that When attached to the at least one arm the free end of the at least one downward protrusion is in about the same, horizontal line as the free end of the arm. In some embodiments, two downward protrusions may be formed and attached to each arm of a plurality of arms. One of the at least one downward protrusions may be attached to the arched arm at a positon so that it is adjacent and in a vertical line with the proximal opening of the tubular member on is the at least one arm is fixed. The second downward protrusion may be attached to the arched arm at a position so that it will be adjacent and in a vertical line with the proximal opening of the corresponding tubular member onto which the arched arm hooks after the one tubular member is connected to the other tubular member of a pair of tubular members.

The method may include attaching One end of a plurality of arched arms in spaced apart relation about the distal extremity of one of the hollow tubular members facilitating a plurality of arms arched from the distal extremity and extending over the first proximal extremity of the hollow tubular member 534. The attaching may be done using a technique, such as welding or gluing. In some embodiments, the at least one arm is integrally formed with the tubular member and is constructed from an extension of the tubular member.

In one embodiment, the method may include forming on the tubular member is the fixed arched arms corresponding attachment means, which correspond to the plurality of arched arms connection means. The method may include forming a plurality of spaced apart slots from a suitable material on the tubular member without the plurality of fixed arched arms. In one embodiment, the plurality of slots may be formed adjacent to the distal opening of the tubular member without the plurality of fixed arms. The plurality of slots may be disposed within the cavity of the tubular member without the plurality of fixed arms. In an alternative embodiment, the plurality of slots may be formed on a suitable position on the body of the tubular member without the plurality of fixed arms. The number of slots formed may correspond to the number of arched arms. Each of the plurality of slots may be sized to accommodate the at least one protrusion of the arched arm.

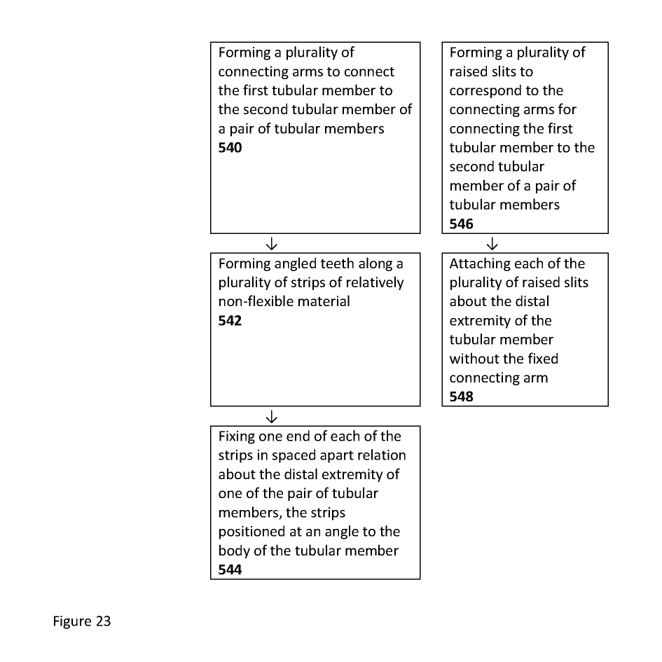

The present invention provides a method of production of alternative connection means for connecting one tubular member to the other tubular member of a pair of tubular members. FIG. 23 shows a flow chart of an exemplary method of constructing the connection means according to an aspect of the present invention.

In one embodiment forming a plurality of connection means may include forming a plurality of arms 540. The plurality of arms may include linear arm extensions featuring ratchet attachment, means. Each linear arm may be formed from a strip of relatively non-flexible material. A plurality of angled teeth may be formed in spaced relation along the strip 542.

The method may include attaching one end of a plurality of the arms about the distal extremity of one of the hollow tubular members facilitating a plurality of arms extending from the distal extremity and over the first proximal extremity of the hollow tubular member 544. Each of the plurality of arms is attached spaced apart on the circumference of the tubular member. The method may include forming a plurality of raised slits 546. The method may include attaching the plurality of raises slits in spaced apart relation about the distal extremity of the corresponding tubular member without the fixed plurality of arms 548. The positioning and sizing of the plurality of arms and the plurality of slits is determined so that each of the plurality of arms is positioned at a higher elevation than the corresponding raised slit, but wherein the flexibility of the arm will allow the arm to be pushed down to the same height as the corresponding slit. The attaching of the at least one arms and. the at least one raised slits may be done using a technique, such as welding or gluing. In some embodiments, the at least one arm is integrally formed with the tubular member and is constructed from an extension of the tubular member. In some embodiments, the at least one raised slit is integrally formed with the tubular member without the plurality of fixed arms and is constructed from an extension of the tubular member.

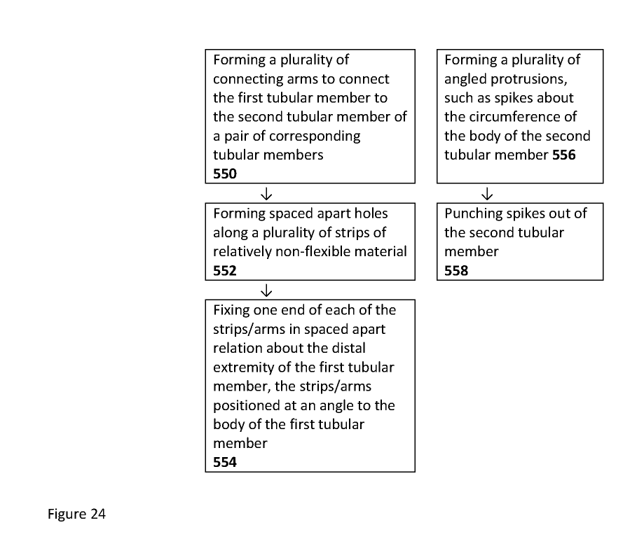

The present invention provides a method of production of connection means featuring a strip of spaced apart openings for connecting one tubular member to the other tubular member of a pair of tubular member. FIG. 24 shows a flow chart of an exemplary method of constructing the connection means according to an aspect of the present invention.

In one embodiment forming a plurality of connection means may include forming a plurality of arms 550. The plurality of arms may include linear arm extensions including spaced apart holes. The openings, such as holes may be formed by any suitable method of making holes along each strip of a plurality of strips of relatively rigid material 552.

The method may include fixing one end of each of the plurality of arms in spaced apart relation about the distal extremity of one of the hollow tubular members facilitating a plurality of arms extending from the distal extremity and over the first proximal extremity of the hollow tubular member 554. Each of the plurality of arms is attached spaced apart on the circumference of the tubular member. The plurality of arms may be fixed to the tubular member to provide arms extending, at an angle to the body of the tubular member. In one embodiment the method may include forming a plurality of protrusions on the corresponding tubular member without the fixed plurality of arms 556. The protrusions may be spikes, which may be made by punching spikes out of the tubular member as described herein for the spikes used for eversion of biological structures and non-biological structures to be joined in the device of the present invention 558. The positioning and sizing of the plurality of arms and the plurality of protrusions is determined so that each of the plurality of arms is positioned at a lower elevation than the corresponding raised and angled protrusion, but wherein the flexibility of the arm will allow the arm to be pushed upwards so that the free end of the spike can be inserted in a hole of the arm. The spike is angled so that after insertion it cannot be displaced out of the hole. The attaching of the at least one arms and the at least one protrusions to the tubular members may be done using a technique, such as welding or gluing. In some embodiments, the at least one arm is integrally formed with the tubular member and is constructed from an extension of the tubular member.

The device and components, such as the tubular members may be sized according to the end use of the device. Different sizes of device may be made for different uses, such as for use with different aged patients, different sex of patients, different types of biological structures, use in people, use in different animals and different sizes of structure to be joined. At least one coating, such as but not limited to a glue may be applied to the device. The material of the tubular membranes may be precoated at any suitable stage. The device may be packaged in a suitable sterile packaging, which may be sealed. The order of the steps of the method is not meant to be limiting and may be in any suitable order.

One skilled in the art can appreciate from the foregoing description that the broad devices and techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

What is claimed is:

1. A device for connection of a first structure with at least one other structure, the device comprising:
a first tubular member, the first tubular member comprising:
a proximal opening at a proximal extremity of the tubular member,
a distal opening at a distal extremity of the tubular member; and
a central cavity within the body of the tubular member for accommodating the first structure; and
a second tubular member, corresponding to the first tubular member, the second tubular member comprising:
a proximal opening at a proximal extremity of the tubular member,
a distal opening at a distal extremity of the tubular member;
a central cavity within the body of the tubular member; and
at least one arm for connecting the first tubular member to the second tubular member,
wherein at least one of the first structure and the at least one other structure is a biological structure.

2-5. (canceled)

6. The device of claim 1, wherein the central cavity of the second tubular member is for accommodating a length of the at least one other structure to be connected to the first structure and wherein the first tubular member and the second tubular member comprise a plurality of spaced apart protrusions on the body of the tubular member for holding an end of a structure everted over the proximal extremity of the tubular member and wherein the plurality of spaced apart protrusions are adjacent to the proximal extremity of the tubular member and protrude outwards away from an exterior surface of the tubular member and in a direction towards the distal extremity of the tubular member.

7-9. (canceled)

10. The device of claim 1, wherein the at least one arm comprises an arcuate arm.

11. The device of claim 1, wherein the at least one arm comprises an extension, the extension comprising a first end and a second end and wherein the at least one arm is freely attached to one of the first tubular member and the second tubular member by attachment of the first end of the at least one arm to an attachment point adjacent to the distal extremity of the one of the first tubular member and the second tubular member, to facilitate a tubular member with at least one arm, such that the extension and the second end of the extension of the at least one arm is restrictably moveable about the attached first end at the attachment point.

12-13. (canceled)

14. The device of claim 11, wherein a plurality of the at least one arms are attached in spaced relation about the circumference of the distal extremity of the one of the first tubular member and the second tubular member. cm 15. (canceled)

16. The device of claim 11, wherein the second end of the at least one arm includes at least one attachment member adapted to attach the first tubular member to the second tubular member and wherein the at least one arm comprises an arch and the attachment member comprises a hook positioned at the free end of the at least one arm, the hook comprising at least one prong positioned at an angle to the arch to facilitate hooking the distal end of the corresponding other tubular member of the one of the first tubular member and the second tubular member of the device.

17. The device of claim 16, wherein the at least one prong comprises at least one protrusion and wherein the at least one prong, comprises a length of prong, a first end of the prong fixed to the free end of the at least one arm and a free end of the prong, the at least one protrusion comprising a first at least one protrusion positioned at the free end of the at least one prong, the first at least one protrusion, protruding at an angle to the length of prong, angled towards the first attached end of the arm and configured to hook the distal end of the corresponding other tubular member of the one of the first tubular member and the second tubular member of the device and to attach into the cavity of the corresponding other tubular member to detachably fix the moveable arm to the corresponding other tubular member of the device and wherein the at least one protrusion comprises a second at least one protrusion, the second at least one protrusion protruding at an angle to the length of the prong, angled towards the first attached end of the arm, the second at least one protrusion protruding from a position along the length of the prongs apart from the first at least one protrusion, wherein the second at least one protrusion is longer than the first at least one protrusion and is configured to allow the first at least one protrusion to attach into the cavity of the corresponding tubular member of the device and to prevent displacement of an additional part of the arm from entering the cavity of the corresponding other tubular member and wherein the corresponding other tubular member comprises a corresponding at least one attachment means for attaching the at least one arm attachment members of the one of the first tubular member and the second tubular member with the fixed arm attachment member, the corresponding at least one attachment means comprising at least one of a portion of the cavity of the other tubular member, the distal edge of the other tubular member and a slot/s.

18-21. (canceled)

22. The device of claim 16, wherein the flexibility of the arch facilitates a diameter equal to at least the combined length of the first tubular member and the length of the second tubular member and the length of everted structures protruding over the proximal extremity of the first tubular member and the second tubular member and the arm is adapted to attach to the corresponding tubular member by the tension of the arch.

23. The device of claim 16, wherein the at least one arm comprises at least one downward protruding member adapted to prevent non-unitary displacement of at least one of the first tubular member and the second tubular member in a unitary device, the unitary device comprising a connected first tubular member and second tubular member.

24. The device of claim 11, wherein the second end of the at least one arm includes at least one attachment member adapted to attach the first tubular member to the second tubular member and wherein the at least one arm comprises a plurality of arms and an arm of the plurality of arms comprises a linear extension, the at least one attachment member comprising a ratchet means disposed along the length of the arm extension.

25. (canceled)

26. The device of claim 24, wherein the plurality of arms are fixed to the one of the first tubular member and the second tubular member to facilitate the plurality of arms inclined at an angle away from the body of the one of the first tubular member and the second tubular member in a direction towards the proximal end of the one of the first tubular member and the second tubular member, the plurality of arms overhanging the proximal end of the one of the first tubular member and the second tubular member.

27. The device of claim 26, wherein the other of the first tubular member and second tubular member to which the plurality of arms is not fixed, comprises a plurality of corresponding means to the at least one attachment member, the plurality of corresponding means comprising a plurality of raised unidirectional slits and wherein the plurality of unidirectional slits are fixed in spaced relation to the body of the other of the first tubular member and the second tubular member to which the plurality or arms is not fixed, to correspond to a respective arm of the plurality of arms and wherein the plurality of unidirectional slits are elevated to a height below the height of the respective arm and wherein each of the plurality of elevated slits are configured for receiving one of the plurality of arms through the slit and for preventing the arm from reverse displacement out of the slit and for promoting the arm to proceed through the slit until the proximal opening of the first tubular member and the proximal opening of the second tubular member or structures everted thereon are in contact.

28-29. (canceled)

30. The device of claim 11, wherein the second end of the at least one arm includes at least one attachment member adapted to attach the first tubular member to the second tubular member and wherein the at least one arm comprises a plurality of arms and an arm of the plurality of arms comprises a linear extension, the at least one attachment member comprising a plurality of openings disposed spaced apart along the length of the linear extension.

31. The device of claim 30, wherein the plurality of arms are fixed to the one of the first tubular member and the second tubular member to facilitate the plurality of arms inclined at an angle away from the body of the tubular member with the at least one arm in a direction towards the proximal end of the tubular member with the at least one arm and overhanging the proximal end of the tubular member with at least one arm and wherein the other of the first tubular member and second tubular member to which the plurality of arms is not fixed, comprises a plurality of corresponding means to the at least one attachment member, the plurality of corresponding means comprising a plurality of protrusions spaced apart and fixed on the body of the other tubular member, the plurality of protrusions configured to fit into an opening of the plurality of openings on the linear extension of the arm and angled to prevent reverse displacement out of the opening and wherein the plurality of protrusions are fixed such that the free ends of the plurality of protrusions are positioned at an elevated height with respect to the corresponding arm of the plurality of arms.

32-36. (canceled)

37. The device of claim 1, wherein the first tubular member and the second tubular member are at least one of split rings comprising a length of tubular member configured in a tubular confirmation with a longitudinal split or a length of the tubular member furled to facilitate a reversibly expandable central cavity of the tubular member, the central cavity configured without a gap, complete rings and a plurality of interwoven lengths configured for reversible radial enlargement of the tubular member when a pushing force is applied to at least one end of the tubular member and reversible radial contraction when a pulling force is applied to at least one end of the tubular member.

38-53. (canceled)

54. A system for performing a bypass procedure to connect a graft to a first opening in an artery before a blockage and to a second opening in an artery after a blockage, the system comprising:
a first device for joining the first opening in the artery to a first end of the graft, the first device comprising:
a first tubular member, the first tubular member comprising:
a proximal opening at a proximal extremity of the tubular member;
a distal opening at a distal extremity of the tubular member;
a central cavity within the tubular member for accommodating the graft; and
a plurality of spaced apart protrusions for holding the first end of the graft over the proximal extremity of the tubular member;
a second tubular member corresponding to the first tubular member, the second tubular member comprising:
a proximal opening at a proximal extremity of the tubular member,
a distal opening at a distal extremity of the tubular member;
central cavity within the tubular member; and
a plurality of spaced apart protrusions for holding an edge of the first opening in the artery everted over the proximal extremity of the tubular member; and
at least one arm for connecting the first tubular member to the second tubular member;
wherein the proximal opening of the first tubular member corresponds to the proximal opening of the second tubular member and wherein the proximal opening of the first tubular member and the proximal opening of the second tubular member are configured for an end to side connection; and
a second device for joining the second end of the graft to the second opening in the artery, the second device comprising:
a first tubular member, the first tubular member comprising:
a proximal opening at a proximal extremity of the tubular member;
a distal opening at a distal extremity of the tubular member;
a central cavity within the tubular member for accommodating the graft; and
a plurality of spaced apart protrusions for holding the second end of the graft everted over the proximal extremity of the tubular member;
a second tubular member corresponding to the first tubular member, the second tubular member comprising:
a proximal opening at a proximal extremity of the tubular member,
a distal opening at a distal extremity of the tubular member;
a central cavity within the tubular member; and
a plurality of spaced apart protrusions for holding an edge of the second opening in the artery everted over the proximal extremity of the tubular member; and
at least one arm for connecting the first tubular member to the second tubular member;
wherein the proximal opening of the first tubular member corresponds to the proximal opening of the second tubular member and wherein the proximal opening of the first tubular member and the proximal opening of the second tubular member are configured for an end to side connection.

55. (canceled)

56. A method of connecting a first biological structure and a second at least one other structure, the method comprising:
providing a first device comprising:
a first tubular member and a second tubular member, the first tubular member comprising:
a proximal opening at a proximal extremity of the tubular member;
a distal opening at a distal extremity of the tubular member;
a central cavity within the tubular member for accommodating the first structure;
a plurality of spaced apart protrusions for holding an end of the first structure everted over the proximal extremity of the tubular member; and
at least one arm fixedly attached to the first tubular member;
the second tubular member comprising:
a proximal opening at a proximal extremity of the tubular member;
a distal opening at a distal extremity of the tubular member;
a central cavity within the tubular member for accommodating the second other structure;
a plurality of spaced apart protrusions for holding an end of the second other structure everted over the proximal extremity of the tubular member;
inserting into the cavity of the first tubular member, through the distal opening, the first biological structure or a part thereof;
everting the end of the first biological structure or part thereof over the opening at the proximal extremity of the first tubular member;
fixing the everted end of the first biological structure or part thereof with the plurality of spaced apart protrusions on the first tubular member;
inserting into the cavity, through the distal opening of the second tubular member, the second other structure to be joined to the first biological structure;
everting the end of the second structure over the opening at the proximal extremity of the second tubular member;
fixing the everted end of the second structure with the plurality of spaced apart protrusions on the second tubular member;
contacting end to end the everted end of the first biological structure with the everted end of the second structure; and
manipulating the at least one arm to attach to a corresponding means on the distal extremity of the second tubular member to connect the first tubular member to the second tubular member and to connect the end of the first biological structure with the end of the second structure.

57-58. (canceled)

59. The method of claim 56, wherein the manipulating comprises inserting a hook of the at least one arm through the distal opening of the second tubular member and into the cavity of the second tubular member.

60. The method of claim 56, wherein the manipulating comprises inserting a toothed extension of the at least one arm through the corresponding means comprising at least one unidirectional slit fixed on the second tubular member and wherein the manipulating comprises pushing the at least one arm downwards to insert the toothed extension of the at least one arm through the at least one unidirectional slit, which is at a lower elevation than the toothed extension of the at least one arm.

61. (canceled)

62. The method of claim 56, wherein the corresponding means comprises at least one spike and the manipulating comprises inserting the at least one spike fixed on the second tubular member into an opening of a plurality of spaced apart openings disposed along the length of the at least one arm.

63-77. (canceled)
